(19) United States
(12) Patent Application Publication
(10) Pub. No.: US 2023/0130831 A1
(43) Pub. Date: Apr. 27, 2023

(54) METHODS FOR DETECTING AND TREATING JOINT DISEASE

(71) Applicant: Washington University, St. Louis, MO (US)

(72) Inventors: Samuel ACHILEFU, St. Louis, MO (US); Christine PHAM, St. Louis, MO (US); Shaw-Wei TSEN, St. Louis, MO (US)

(21) Appl. No.: 18/049,504

(22) Filed: Oct. 25, 2022

Related U.S. Application Data
(60) Provisional application No. 63/271,783, filed on Oct. 26, 2021.

(51) Int. Cl. A61K 49/00; A61K 31/519; A61K 31/573; A61P 19/02

(52) U.S. Cl. CPC A61K 49/0056; A61K 49/0032; A61K 31/519; A61K 31/573; A61P 19/02

(57) ABSTRACT

The present disclosure provides a method of detecting a joint disease, the method comprising administering a composition comprising an agent chosen from LS301, LS838, or a derivative thereof to a subject in need thereof; and detecting a signal intensity emitted from the agent in at least one joint of the subject, wherein detection of signal intensity above a baseline value indicates the subject has the joint disease. Also are provided are methods for monitoring the progression of a joint disease, monitoring treatment response to a therapeutic agent for treating a joint disease, and treating a joint disease.

### CROSS-REFERENCE

This application claims the benefit of priority of U.S. Provisional Patent Application Ser. No. 63/271,783 filed Oct. 26, 2021, the disclosure of which is incorporated by reference in its entirety for all purposes.

### GOVERNMENT LICENSE RIGHTS

This work was supported by the US Department of Veterans Affairs, and the Federal Government has certain rights in this invention.

### TECHNICAL FIELD

The present disclosure generally relates to the detection and treatment of joint diseases.

### SUMMARY

Among the various aspects of the present disclosure is the provision of methods for detecting and treating joint diseases.

The present disclosure provides a method of detecting a joint disease, the method comprising administering a composition comprising an effective amount of LS301, LS838, or a derivative thereof to a subject and detecting a signal intensity emitted from the agent in at least one joint of the subject, wherein detection of signal intensity above a baseline value indicates the subject has arthritis.

The present disclosure also provides a method for monitoring disease progression of a joint disease, the method comprising administering a composition comprising an effective amount of LS301, LS838, or a derivative thereof to a subject, detecting a first signal intensity emitted from the agent in at least one joint of the subject at a first time point, and detecting a second signal intensity emitted from the agent in at least one joint of the subject at a second time point, wherein if the first signal intensity is greater than the second signal intensity, the disease is determined to be regressing, or if the first signal intensity is less than the second signal intensity, the disease is determined to be progressing.

The present disclosure further provides a method of monitoring treatment response to a therapeutic agent for treating a joint disease in a subject, the method comprising (i) administering a composition comprising an effective amount of LS301, LS838, or a derivative thereof to a subject, (ii) detecting a first signal intensity emitted from the agent in at least one joint of the subject at a first time point, (iii) administering a therapeutic agent to the subject, (iv) repeating step (i), (v) detecting a second signal intensity emitted from the agent in at least one joint of the subject at a second time point, and (vi) comparing the second signal intensity to the first signal intensity.

The present disclosure provides a method of treating a joint disease in a subject, the method comprising administering a composition comprising an effective amount of an LS301 derivative or an LS838 derivative thereof the subject.

Other objects and features will be in part apparent and in part pointed out below.

### DESCRIPTION OF THE DRAWINGS

The patent or application file contains at least one drawing executed in color. Copies of this patent or patent application publication with color drawing(s) will be provided by the Office upon request and payment of the necessary fee.

Those skilled in the art will understand that the drawings described below are for illustrative purposes only. The drawings are not intended to limit the scope of the present teachings in any way.

FIG. 1(A-F) shows in-vivo imaging of K/BxN spontaneous and serum transfer arthritis using LS301. FIG. 1A shows early-stage spontaneous K/BxN arthritis (3-4 weeks old F1 mice).

FIG. 1B shows intermediate stage spontaneous K/BxN arthritis (5-7 weeks old F1 mice). FIG. 1C shows late-stage spontaneous K/BxN arthritis (9-10 weeks old F1 mice). FIG. 1D shows C57BL/6J mice with serum transfer arthritis (day 4 post disease induction). FIG. 1E shows normal (control) C57BL/6J mice injected with LS301. FIG. 1F shows intermediate stage spontaneous K/BxN arthritis (5-7 weeks old) F1 mice injected with cypate dye (control) in lieu of LS301.

FIG. 2(A-B) shows typical LS301 biodistribution in arthritic mice. FIG. 2A shows an example ex-vivo organ biodistribution of LS301 in C57BL/6J mice with serum transfer arthritis (left) or in control C57BL/6J mice (right) 18 hours after intravenous injection, as assessed by near-infrared fluorescence imaging on the Pearl animal imaging system. FIG. 2B quantifies fluorescence from individual organs from FIG. 2A (n=4 mice per group).

FIG. 3(A-B) correlates LS301 fluorescent signal with disease severity in individual limbs. C57BL/6 mice (n=4 per group) with serum transfer arthritis (day 4 post disease induction) were injected intravenously with 6 nmol LS301. Fluorescence was plotted against clinical scores (FIG. 3A) and ankle thickness measurements (FIG. 3B).

FIG. 4(A-C) monitors arthritis disease remission using LS301. FIG. 4A shows the representative fluorescence images of arthritic mice imaged with LS301 at disease day 4 (left) and after disease remission (right). FIG. 4B shows the comparison of average total extremity fluorescence (quantitated from ROIs) per mouse described in FIG. 4A. FIG. 4C shows the total paw arthritis scores and change in ankle thickness from baseline of mice described in FIG. 4A.

FIG. 5(A-D) monitors DEX-associated treatment response using LS301. Mice in FIG. 5A remained untreated as controls or FIG. 5B were treated with daily doses of intraperitoneal dexamethasone (10 mg/kg/dose) over 6 days. FIG. 5C compares average total extremity fluorescence (quantitated from ROIs) per mouse described in FIGS. 5A and 5B. FIG. 5D shows total paw arthritis scores and change in ankle thickness from baseline of mice described in FIGS. 5A and 5B.

FIG. 6(A-D) shows the cellular localization of LS301 in the arthritic mouse paws. FIG. 6A shows an example of Pearl near-infrared fluorescence images of mice immediately before limb harvest. FIG. 6B(1-2) H&E and fluorescence images from corresponding regions of articular cartilage in mouse ankle. FIG. 6C shows fluorescence microscopy images from corresponding regions of articular cartilage in mouse ankle. Brief description of FIG. 6D needed here.

FIG. 7(A-B) shows LS301 association with cartilage damage. K/BxN F1 mice (6-7 weeks old) (n=2 mice per group) were injected intravenously with 6 nmol LS301. FIG. 7A shows images from regions associated with pannus/bone erosion. Arrows indicate areas of LS301 accumulation. FIG. 7B shows images with preferential association of LS301 at regions of cartilage damage (loss of Safranin 0 staining).

FIG. 8(A-E) shows imaging of rheumatoid arthritis (RA) using LS301. FIG. 8A shows early stage RA (3-4 week old K/Bxn mice). FIG. 8B shows Intermediate stage RA (3-4 week old K/Bxn mice). FIG. 8C shows late-stage RA (9-10 week old K/Bxn mice). FIG. 8D shows normal (control) C57BL/6J mice. FIG. 8E shows K/Bxn mice (3-4 weeks old) were injected with cypate dye (control) instead of LS301.

FIG. 9 shows the kinetics of in-vivo LS301 clearance.

FIG. 10A and FIG. 10B show typical LS301 biodistribution in arthritic mice.

FIG. 11 shows imaging arthritis using subcutaneously administered LS301.

FIG. 12 monitoring arthritis disease remission using LS301.

FIG. 13 shows the in-vitro anti-inflammatory effect of LS301-methotrexate (MTX) derivative, or LS301 combined with laser irradiation, vs. MTX alone and controls.

FIG. 14 shows the in-vivo therapeutic efficacy of LS301-MTX derivative vs. LS301 alone or control.

### DETAILED DESCRIPTION

Rheumatoid arthritis (RA) is among the most common debilitating joint conditions in the United States, affecting up to 1% of the population. In recent decades, therapeutic advances in disease-modifying antirheumatic drugs (DMARDs) have inhibited disease progression and made clinical remission an achievable goal. However, a challenge remains in optimizing treatment regimens to reach such a state in the shortest time to minimize damage to the joints caused by elevated disease activity.

There is currently a lack of methods for monitoring early treatment response in RA patients, which has hampered the accurate assessment of disease activity and posed a significant barrier to treatment adjustment. In the context of RA, successful clinical management relies on the proper selection of a therapy to which the patient will show a response.

Drug responses vary among patients, so no well-established methods guide therapeutic choices. The current clinical paradigm involves a series of trials and errors, where treatment response is monitored over months relying on clinical scoring methods and imaging modalities such as X-rays and ultrasonography, which are insensitive to very early (i.e., <1 month) changes in disease activity. Positron emission tomography (PET), an experimental approach for arthritis imaging, provides greater sensitivity but has the disadvantage of increased radiation exposure. Together, these limitations lead to additional costs and toxicities to the patient and potentially worsen patient outcomes since early treatment is ideal for optimal reduction in joint damage. Furthermore, the speed with which arthritis therapies can be evaluated in clinical trials depends on feedback regarding treatment efficacy. In these regards, there is a need for a noninvasive method that enables rapid assessment of therapeutic effects for RA.

Recently, fluorescence imaging (FI) has been explored to diagnose and track arthritis treatment response in preclinical and human models. The technique involves administering a fluorophore, then detecting accumulated fluorescence in affected joints. FI has several advantages over conventional imaging modalities, including its low cost and the avoidance of ionizing radiation exposure. FI is well-suited for application to RA, where small peripheral joints in the extremities are involved. Agents used in prior studies include non-targeted dyes (e.g., indocyanine green (ICG), Cy5.5); dye-labeled monoclonal antibodies and small molecule ligands that bind macrophage or endothelial cell targets such as F4/80, E-selectin, αvβ3 integrin, and folate receptors; and enzyme-activatable probes. Non-targeted dyes such as ICG accumulate in inflamed joints primarily due to increased vascular permeability, resulting in low contrast compared to targeted agents. On the other hand, the existing targeted approaches which focus on activated macrophages and endothelial cells have reduced specificity as these cell types are not limited to RA.

Early treatment response monitoring and targeted therapy of rheumatoid arthritis (RA) remain a challenge in the clinic. Described herein is a system for monitoring of drug treatment response via infrared fluorescence and selective targeting of disease sites in RA using LS301.

As shown herein, (1) LS301 selectively accumulates in arthritic joints in animal models of RA; (2) LS301 fluorescence correlates with arthritis disease severity; (3) LS301 can be used to track RA disease remission after treatment; and (4) combining LS301 with photodynamic laser irradiation or covalently linked RA drug methotrexate leads to therapeutic effect against RA (see Examples 1 and 2). Therefore, LS838 is expected to behave similarly to LS301 in these systems.

This system may be potentially developed into a portable technology for imaging and treating joints in extremities, including the hands (a primary disease site in RA), feet, ankles, and knees, which can be used in outpatient settings.

This system overcomes the limitations of the current RA imaging methods, such as conventional X-rays and ultrasound, to provide specific early treatment response information to clinicians regarding disease severity and location. Furthermore, the system serves as a theragnostic technology wherein combining the contrast agent with external laser irradiation or attached drug moieties result in new and improved therapeutic options for patients. With its continued development, it is anticipated that this system will apply to other autoimmune and inflammatory diseases such as lupus, multiple sclerosis, and so forth.

LS301 and LS838

The present disclosure relates to a dye-peptide derivative (LS301) that selectively targets tyrosine 23-phosphorylated annexin A2 (pANXA2) and accumulates in regions of joint pathology in rheumatoid arthritis (RA). As shown herein, LS301 can be used as an imaging agent to track RA disease progression, monitor response to treatment, or as a therapeutic for treating RA.

The agent can be LS301 or an analog that selectively targets tyrosine 23-phosphorylated annexin A2 (pANXA2) and accumulates in regions of joint pathology in rheumatoid arthritis (RA).

In certain embodiments, the cypate is

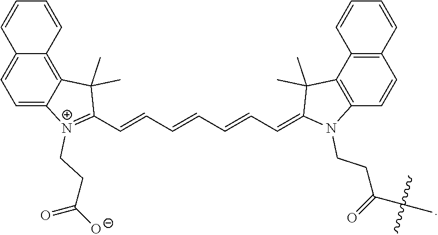 [SMILES: *C(=O)CCN1/C(=C/C=C/C=C/C=C/C2=[N+](CCC(=O)[O-])c3ccc4ccccc4c3C2(C)C)C(C)(C)c2c1ccc1ccccc21]

In certain embodiments, at least one of the Cys amino acid residues is D-Cys.

In certain embodiments, the compound is LS301 and comprises the structural formula

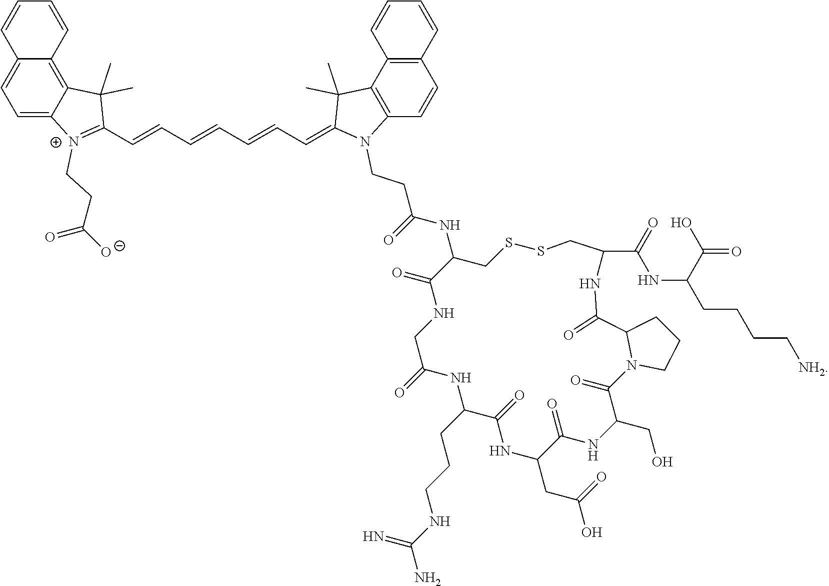 [SMILES: CC1(C)C(/C=C/C=C/C=C/C=C2/N(CCC(=O)NC3CSSCC(C(=O)NC(CCCCN)C(=O)O)NC(=O)C4CCCN4C(=O)C(CO)NC(=O)C(CC(=O)O)NC(=O)C(CCCNC(=N)N)NC(=O)CNC3=O)c3ccc4ccccc4c3C2(C)C)=[N+](CCC(=O)[O-])c2ccc3ccccc3c21]

In certain embodiments, the compound is LS301 comprising the structural formula

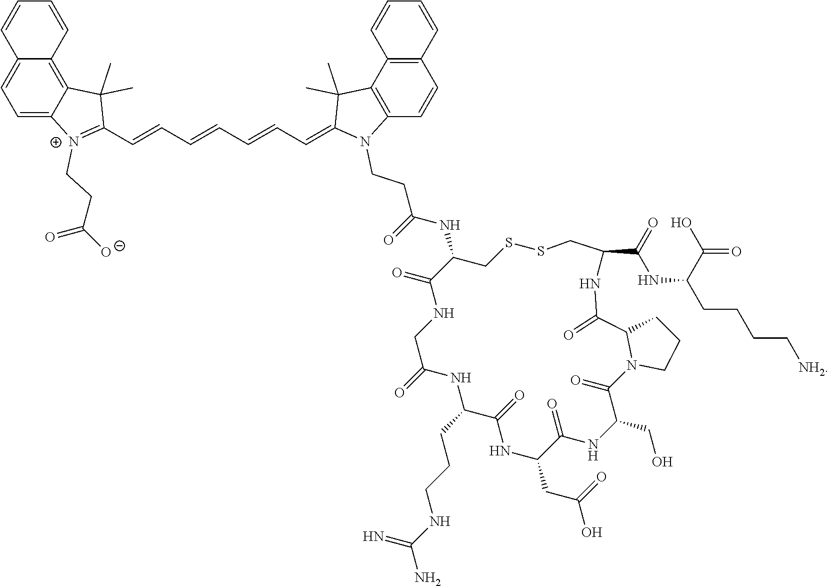

In certain embodiments, a dye-conjugate chosen from cypate-Cyclo(Cys-Gly-Arg-Asp-Ser-Pro-Cys)-Lys-OH (LS301), cypate-Cyclo(Cys-Gly-Arg-Asp-Ser-Pro-Cys)-Tyr-OH (LS838) or pharmaceutically acceptable salts thereof.

In certain embodiments, the pharmaceutical composition comprises the compound LS838, a derivative of LS301. In certain embodiments, LS838 comprises the structural formula

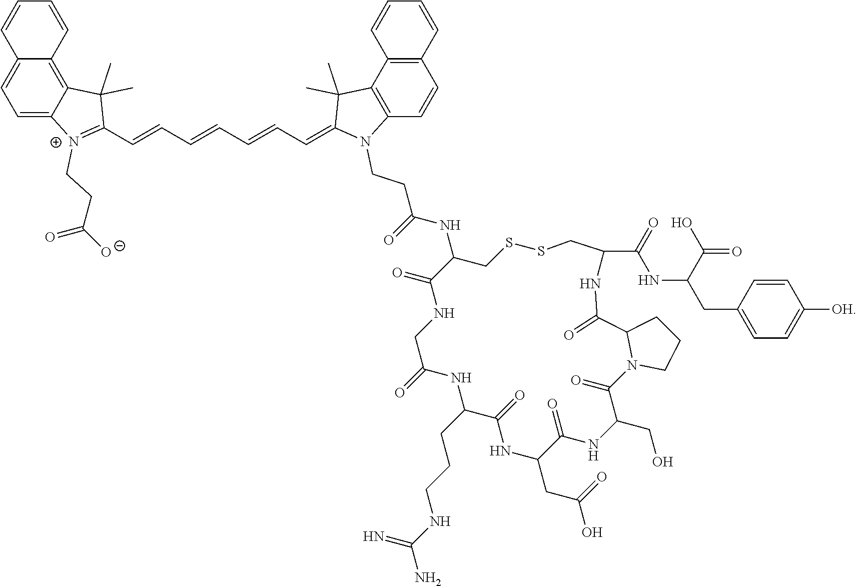 [SMILES: CC1(C)C(/C=C/C=C/C=C/C=C2/N(CCC(=O)NC3CSSCC(C(=O)NC(Cc4ccc(O)cc4)C(=O)O)NC(=O)C4CCCN4C(=O)C(CO)NC(=O)C(CC(=O)O)NC(=O)C(CCCNC(=N)N)NC(=O)CNC3=O)c3ccc4ccccc4c3C2(C)C)=[N+](CCC(=O)[O-])c2ccc3ccccc3c21]

In certain embodiments, the compound is LS838 and comprises the structural formula

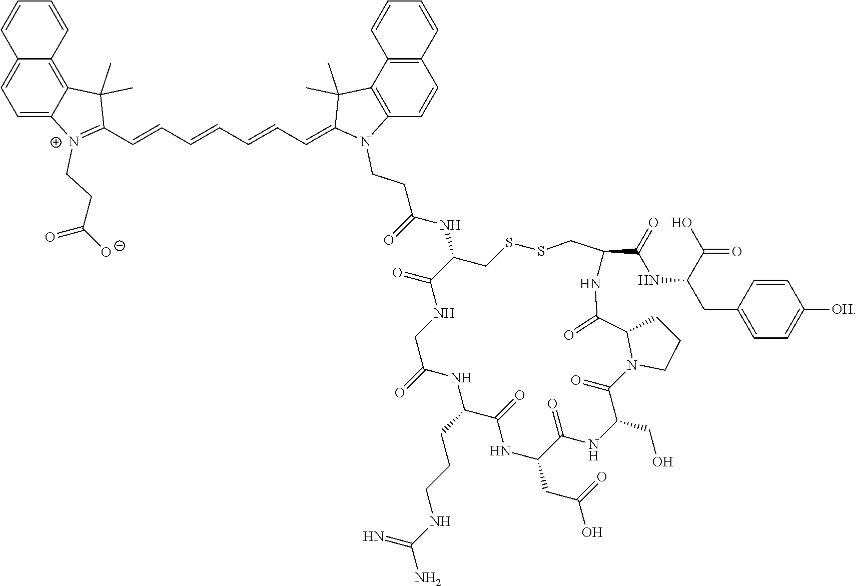 [SMILES: CC1(C)C(/C=C/C=C/C=C/C=C2/N(CCC(=O)N[C@@H]3CSSC[C@@H](C(=O)N[C@@H](Cc4ccc(O)cc4)C(=O)O)NC(=O)[C@@H]4CCCN4C(=O)[C@H](CO)NC(=O)[C@H](CC(=O)O)NC(=O)[C@H](CCCNC(=N)N)NC(=O)CNC3=O)c3ccc4ccccc4c3C2(C)C)=[N+](CCC(=O)[O-])c2ccc3ccccc3c21]

Without wishing to be bound by theory, the placement of the tyrosine in the LS838 molecule is important for retaining LS838 in tumors. Unlike LS301, LS838 can be radiolabeled at its tyrosine residue, enabling combined intravital fluorescence microscopy and noninvasive imaging. In certain embodiments, the radionuclide is chosen from, for example, fluorine-18, iodine-123, iodine-124, iodine-125, and iodine-131. This radiolabeling allows imaging of cancer in the human body noninvasively using nuclear imaging methods. The fluorescence allows optical methods to guide tissue biopsy, surgery, and assessment of surgical margins.

Derivatives of LS301 and LS838

The methods described herein may use an LS301 derivative or an LS838 derivative. In certain embodiments, the derivative comprises at least one imaging agent or at least one treatment agent. In one embodiment, the derivative comprises imaging agent. In certain embodiments, the derivative comprises an imaging agent and a treatment agent. Irrespective of the embodiment, the derivative may be conjugated to the cyclic peptide directly via a covalent bond or indirectly via a linker.

Several imaging agents are suitable for use to the extent that they provide the ability to detect or monitor the localization of the derivative. In one embodiment, the imaging agent comprises an optical imaging agent. Suitable optical imaging agents include fluorophores, organic fluorescent dyes, luminescent imaging agents, fluorescent lanthanide complexes, and fluorescent semiconductor nanocrystals.

The compounds and derivatives described herein may operate along the electromagnetic spectrum, including visible and infrared wavelengths, such as near-infrared (NIR), short wavelength infrared (SWIR), middle wavelength infrared (MWIR), long wavelength infrared (LWIR), and far-infrared. SWIR has the advantage over NIR of deeper tissue penetration.

Examples of suitable visible (400-700 nm) fluorescent dyes include fluorescein, FITC, rhodamine, Texas Red, CyDyes (e.g., Cy3, Cy5, Cy5.5), Alexa Fluors (e.g., Alexa⁴⁸⁸, Alexa⁵⁵⁵, Alexa⁵⁹⁴; Alexa⁶⁴⁷) and DyDelight Dyes. Suitable near-infrared (NIR) (700-1400 nm) fluorescent dyes include carbocyanine dyes, such as cypate and its derivatives. Luminescence imaging agents include luminescent lanthanide chelates and bioluminescence compounds (e.g., bacterial Lux, eukaryotic Luc, or Ruc systems). In a specific embodiment, an imaging agent is a carbocyanine dye or a derivative thereof. Suitable carbocyanine dyes are known in the art. In certain embodiments, the derivative comprises a carbocyanine dye chosen from Cypate (cypate 4), LS288, LS798, LS276, LS843, Cypate 3, and Cypate 2. A derivative comprising a carbocyanine dye may comprise a nonionic group (i.e., polyethylene glycol) or a positively charged moiety (i.e., ⁺NMe₃) conjugated to a free carboxylic acid group of a cypate.

Short-wave infrared (SWIR) (1400-3000 nm) operates at longer wavelength than NIR. Many NIR dyes produce signal in the SWIR albeit weaker than the signal in the NIR range. MWIR is between 3000 nm and 8000 nm (3-8 μm). LWIR is between 8000 and 15000 nm (8-15 μm. Far infrared is between 15 and 1,000 μm. To visualize in these ranges, one of skill in the art would select a dye active at those wavelengths.

Alternatively, a derivative comprising a carbocyanine dye may comprise a functional group for conjugation of a radioisotope, treatment agent, or another biologically active molecule. Non-limiting examples of biologically active molecules include nanoparticles, small organic molecules, peptides, proteins, organometallics, drugs, antibiotics, and carbohydrates. In certain embodiments, the biologically active molecule is <500 Da. In certain embodiments, a functional group is chosen from alkyne, azido (N₃), and a chelating agent. As used herein, a “chelating agent” is a molecule that forms multiple chemical bonds with a single metal atom. Examples of chelating agents include, but are not limited to, iminodicarboxylic and polyaminopolycarboxylic reactive groups, diethylenetriaminepentaacetic acid (DTPA), 1,4,7,10-tetraazacyclododecane-1,4,7,10-tetraacetic acid (DOTA), tetramethyl heptanedionate (TMHD), 2,4-pentanedione, ethylenediamine-tetraacetic acid disodium salt (EDTA), ethyleneglycol-O,O′-bis(2-aminoethyl)-N,N,N′,N′-tetraacetic acid (EGTA), N-(2-hydroxyethyl)ethylenediamine-N,N′,N′-triacetic acid trisodium salt (HEDTA), nitrilotriacetic acid (NTA), and 1,4,8,11-tetraazacyclotetradecane-N,N′,N″,N′″-tetraacetic acid (TETA), deferoxamine (DFO), and derivatives thereof.

An imaging agent emits a signal that can be detected by a signal-transducing machine. In some cases, an imaging agent can emit a signal spontaneously, such as when the detectable label is a radionuclide. In other cases, the imaging agent emits a signal as a result of being stimulated by an external field, such as when the imaging agent is a relaxivity metal. Examples of signals include, without limitation, gamma rays, X-rays, visible light, infrared energy, and radio waves. Non-limiting examples of modalities of imaging may include magnetic resonance imaging (MRI), ultrasound (US), computed tomography (CT), Positron Emission Tomography (PET), Single Photon Emission Computed Tomography (SPECT), Optical coherence tomography (OCT), and optical imaging (OI, bioluminescence, and fluorescence).

In an alternative embodiment, the derivative comprises radiological imaging agent. In certain embodiments, the derivative comprises two imaging agents, for example a carbocyanine dye or derivative thereof and a radioisotope. The radioisotope may be conjugated to the carbocyanine dye or may be conjugated to residue of the peptide, such as Tyr. Many radioisotopes can be detected and are suitable for use herein. Examples of radiological imaging agents include, but are not limited to, antimony-124, antimony-125, arsenic-74, barium-103, barium-140, beryllium-7, bismuth-206, bismuth-207, cadmium-109, cadium-115, calcium-45, cerium-139, cerium-141, cerium-144, cesium-137, chromium-51, gadolinium-153, gold-195, gold-199, hafnium-175-181, indium-111, iridium-192, iron-55, iron-59, krypton-85, lead-210, manganese-54, mercury-197, mercury-203, molybdenum-99, neodymium-147, neptunium-237, nickel-63, niobium-95, osmium-185, palladium-103, platinum-195, praseodymium-143, promethium-147, protactinium-233, radium-226, rhenium-186, rubidium-86, ruthenium-103, ruthenium-106, scandium-44, scandium-46, selenium-75, silver-110, silver-111, sodium-22, strontium-85, strontium-89, strontium-90, sulfur-35, tantalum-182, technetium-99, tellurium-125, tellurium-132, thallium-204, thorium-228, thorium-232, thallium-170, tin-113, titanium-44, tungsten-185, vanadium-48, vanadium-49, ytterbium-169, yttrium-88, yttrium-90, yttrium-91, zinc-65, and zirconium. In a further alternative embodiment, the radiological imaging agent is selected from the group consisting of technetium-99, indium-111, strontium-90, iodine-125, thallium-201, fluorine-18, carbon-11, carbon-13, nitrogen-13, oxygen-15, copper-64, lutetium-177, yttrium-90, and iodine-123, iodine-124, iodine-125, and iodine-131. In certain embodiments, a radioisotope images and treats. It is known in the art that radioisotopes function as both imaging agents and treatment agents. For example, since iodine-131 has both beta and gamma decay modes and can be used for radiotherapy or for imaging. Thus, the derivative may be ¹³¹I-LS301 or ¹³¹I-LS838.

Many other imaging agents are suitable in the derivatives, for example, gadolinium, metalloporphyrin, ferric chloride, ferric ammonium citrate, and ferrioxamine methanesulfonate for magnetic resonance imaging.

The LS301 and LS838 derivatives herein optionally includes one or more treatment agents, such as a drug or hormone. In certain embodiments, the derivative comprises a carbocyanine dye or derivative thereof and a treatment agent. As will be appreciated by the skilled artisan, the choice of a particular treatment agent can and will vary depending upon the indication to be treated and its stage of progression. Because the derivatives herein selectively target cells that express phosphorylated Tyr(23) Annexin A2 (pANXA2), the treatment agents are generally directed toward the treatment of an pANXA2-mediated disorder, such as a joint disease, diabetes, inflammation, cardiovascular disease, and cancer.

For example, when the indication is a joint disease, such as arthritis, the treatment agent may be methotrexate, dexamethasone, hydroxychloroquine, sulfasalazine, leflunomide, adalimumab, rituximab, abatacept, etanercept, anakinra, azathioprine, cyclophosphamide, and cyclosporine. Thus, the derivative may be chosen from LS301-methotrexate, LS301-dexamethasone, LS301-hydroxychloroquine, LS301-sulfasalazine, LS301-leflunomide, LS301-adalimumab, LS301-rituximab, LS301-abatacept, LS301-etanercept, LS301-anakinra, LS301-azathioprine, LS301-cyclophosphamide, LS301-cyclosporine, LS838-methotrexate, LS838-dexamethasone, LS838-hydroxychloroquine, LS838-sulfasalazine, LS838-leflunomide, LS838-adalimumab, LS838-rituximab, LS838-abatacept, LS838-etanercept, LS838-anakinra, LS838-azathioprine, LS838-cyclophosphamide, and LS838-cyclosporine.

A common symptom of joint diseases, including arthritis, is inflammation. Thus, anti-inflammatory agents, such as non-steroidal anti-inflammatory drug (NSAID), and corticosteroids are commonly prescribed. Examples of NSAIDs include, but are not limited to, ibuprofen, aniline derivatives (acetaminophen), indole-3-acetic acid derivatives (indomethacin), specific Cox-2 inhibitors (celecoxib), and aspirin. Thus, the derivative may be chosen from LS301-ibuprofen, LS301-acetaminophen, LS301-indomethacin, LS301-celecoxib, LS301-aspirin, LS838-ibuprofen, LS838-acetaminophen, LS838-indomethacin, LS838-celecoxib, and LS838-aspirin. Suitable corticosteroids include, but are not limited to, betamethasone, dexamethasone, prednisone, methylprednisolone, triamcinolone, cortisone, hydrocortisone, budesonide, beclomethasone, fluticasone, mometasone, and vamorolone. Thus, the derivative may be chosen from LS301-betamethasone, LS301-dexamethasone, LS301-prednisone, LS301-methylprednisolone, LS301-triamcinolone, LS301-cortisone, LS301-hydrocortisone, LS301-budesonide, LS301-beclomethasone, LS301-fluticasone, LS301-mometasone, LS301-vamorolone, LS838-betamethasone, LS838-dexamethasone, LS838-prednisone, LS838-methylprednisolone, LS838-triamcinolone, LS838-cortisone, LS838-hydrocortisone, LS838-budesonide, LS838-beclomethasone, LS838-fluticasone, LS838-mometasone, and LS838-vamorolone.

For example, when the indication is diabetes, the treatment agent may be sulfonylureas, biguanides, thiazolidinediones, meglitinides, D-phenylalanine derivatives, synthetic amylin derivatives, and incretin mimetics. By way of further example, when the indication is cardiovascular disease, the treatment agent may include sodium-channel blockers (e.g., quinidine, ranolazine, phenytoin, disopyramide, lidocaine, mexiletine, triamterene, lamotrigine, amiloride, moricizine, oxcarbazepine, procainamide, tocainide, amiodarone, propafenone, flecainide, encainide, ajmaline, aprindine, tetrodotoxin, eslicarbazepine, pilsicainide, carbamazepine, ethotoin, fosphenytoin, rufinamide, and lacosamide), beta-blockers (e.g., acebutolol, atenolol, betaxolol, bisoprolol, cicloprolol, esmolol, metoprolol, nebivolol, and propranolol), calcium-channel blockers (e.g., agmatine, amiodarone, amlodipine, aranidipine, azelnidipine, barnidipine, bencyclane, benidipine, bepridil, bioallethrin, carboxyamidotriazole, caroverine, carvedilol, cilnidipine, cinnarizine, clevidipine, cyclandelate, darodipine, dexniguldipine, dexverapamil, diltiazem, dotarizine, efonidipine, emopamil, eperisone, ethosuximide, etripamil, fasudil, felodipine, fendiline, flunarizine, fluspirilene, gallopamil, isradipine, lacidipine, lamotrigine, lercanidipine, levamlodipine, levomenthol, lidoflazine, lomerizine, loperamide, manidipine, methsuximide, mibefradil, naftopidil, nicardipine, nifedipine, niguldipine, niludipine, nilvadipine, nimesulide, nimodipine, nisoldipine, nitrendipine, nylidrin, otilonium, penfluridol, perhexiline, pinaverium, prenylamine, seletracetam, terodiline, tetrahydropalmatine, tetrandrine, tranilast, trimebutine, trimethadione, verapamil, vinpocetine, xylometazoline, ziconotide, and zonisamide), diuretics (e.g., hydrochlorothiazide, acetazolamide, amiloride, azosemide, bendroflumethiazide, benzthiazide, brinzolamide, bromotheophylline, bumetanide, buthiazide, canagliflozin, canrenoic acid, canrenone, chlorothiazide, chlorthalidone, cicletanine, clofenamide, clopamide, clorexolone, conivaptan, cyclopenthiazide, cyclothiazide, dapagliflozin, diclofenamide, dorzolamide, drospirenone, efonidipine, empagliflozin, epitizide, eplerenone, ertugliflozin, etacrynic acid, ethoxzolamide, fenquizone, finerenone, furosemide, hydroflumethiazide, ibopamine, indapamide, indisulam, isosorbide, mebutizide, mefruside, mersalyl, methazolamide, methyclothiazide, meticrane, metolazone, muzolimine, piretanide, polythiazide, quinethazone, rolofylline, spiradoline, spironolactone, theobromine, tienilic acid, tolvaptan, torasemide, triamterene, trichlormethiazide, tripamide, ularitide, xipamide, and zonisamide), ACE inhibitors (e.g., benazepril, captopril, cilazapril, delapril, enalapril, fosinopril, lisinopril, moexipril, perindopril, quinapril, ramipril, and zofenopril), and thrombolytic agents (e.g., tissue plasminogen activator and streptokinase). Thus, the derivative may be chosen from these categories above.

In an additional embodiment, when the indication is cancer, the treatment agent may include DNA synthesis inhibitors (e.g., daunorubicin, and adriamycin), mitotic inhibitors (e.g., the taxanes, paclitaxel, and docetaxel), the vinca alkaloids (e.g., vinblastine, vincristine, and vinorelbine), antimetabolites (e.g., 5-fluorouracil, capecitabine, 6-mercaptopurine, methotrexate, gemcitabine, cytarabine (ara-C), fludarabine, pemetrexed, cytosine arabinoside, methotrexate, and aminopterin), alkylating agents (e.g., busulfan, cisplatin, carboplatin, chlorambucil, cyclophosphamide, ifosfamide, dacarbazine (DTIC), mechlorethamine (nitrogen mustard), melphalan, and temozolomide), nitrosoureas (e.g., carmustine (BCNU) and lomustine (CCNU) anthracyclines (e.g., daunorubicin, doxorubicin (Adriamycin), epirubicin, idarubicin, and mitoxantrone), topoisomerase inhibitors (e.g., topotecan, irinotecan, etoposide (VP-16), and teniposide), cytotoxins (e.g., paclitaxel, vinblastine, and macromycin), anti-cytoskeletals (e.g., taxol and colchicine) and angiogenesis inhibitors (e.g., VEGF inhibitors, anti-VEGF Abs). Thus, the derivative may be chosen from these categories above.

Summaries of cancer drugs, including information regarding approved indications, may be found via the National Cancer Institute at the National Institutes of Health (www.cancer.gov/cancertopics/druginfo/alphalist), the FDA Approved Drug Product database (www.accessdata.fda.gov/scripts/cder/drugsatfda/) and the National Comprehensive Cancer Network (NCCN) guidelines (www.nccn.org/professionals/physician_gls/f_guidelines.asp). In a specific embodiment, the derivative may be chemotherapeutic. In certain embodiments, the derivative may be chemotherapeutic for pancreatic cancer.

In certain embodiments, the derivative may comprise hormones (e.g., steroids), antibodies, antibody fragments, peptides, glycopeptides, peptidomimetics, drug mimics, metal chelating agents, radioactive agents, echogenic agents, various drugs (in addition to the ones specifically delineated), antisense molecules, and small inhibitory RNAs.

In certain embodiments, the treatment agent in the derivative may be conjugated to the peptide via one or more linkers. In other embodiments having more than one linear peptide or one or more cyclic peptides, the individual peptides may optionally be conjugated via one or more linkers. Many linkers are suitable. Typically, the linker imparts flexibility to the derivative. Generally speaking, the chain of atoms defining the linker can and will vary depending upon the embodiment.

In certain embodiments, the linker comprises one or more amino acids. Amino acid residue linkers are usually at least one residue and can be 50 or more residues. In an embodiment, a linker may be about 1 to about 10 amino acids. In another embodiment, a linker may be about 10 to about 20 amino acids. In still another embodiment, a linker may be about 20 to about 30 amino acids. In still yet another embodiment, a linker may be about 30 to about 40 amino acids. In different embodiments, a linker may be about 40 to about 50 amino acids. In other embodiments, a linker may be more than 50 amino acids. For instance, a linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids. In a specific embodiment, a linker is 1 amino acid.

Any amino acid residue may be used for the linker. Typical amino acid residues used for linking are glycine, serine, alanine, leucine, lysine, glutamic and aspartic acid, or the like. For example, a linker may be (AAS)ₙ, (AAAL)ₙ, (GₙS)ₙ, or (G₂S)ₙ, wherein A is alanine, S is serine, L is leucine, and G is glycine, and wherein n is an integer from 1-20, or 1-10, or 3-10. Accordingly, n may be 1, 2, 3, 4, 5, 6, 7, 8, 9, 10, 11, 12, 13, 14, 15, 16, 17, 18, 19 or 20. Thus, in certain embodiments, a linker includes, but is not limited to, (AAS)ₙ, (AAAL)ₙ, (GₙS)ₙ, or (G₂S)ₙ, wherein A is alanine, S is serine, L is leucine, and G is glycine and wherein n is an integer from 1-20, or 1-10, or 3-10. In a specific embodiment, a linker is one glycine.

In a further embodiment, the linker comprises hydrocarbyl or substituted hydrocarbyl groups. In certain embodiments, the linker is from about 1 to about 50 atoms in length. Alternatively, the linker is from about 2 about 30 atoms in length. In an embodiment, the linker is from about 4 to about 20 atoms in length. The linker may comprise a variety of heteroatoms that may be saturated or unsaturated, substituted or unsubstituted, linear or cyclic, or straight or branched. The chain of atoms defining the linker will typically be selected from the group consisting of carbon, oxygen, nitrogen, sulfur, selenium, silicon, and phosphorous. In an alternative embodiment, the chain of atoms is selected from the group consisting of carbon, oxygen, nitrogen, sulfur, and selenium. In an embodiment, the linker comprises substantially carbon and oxygen atoms. In addition, the chain of atoms defining the linker may be substituted or unsubstituted with atoms other than hydrogen, including, but not limited to, hydroxy, keto (═O), or acyl, such as acetyl. Thus, the chain may optionally include one or more ether, thioether, selenoether, amide, or amine linkages between hydrocarbyl or substituted hydrocarbyl regions. Exemplary linkers include ethylene glycol and aminohexanoic acid. More specifically, a linker may be a polyethylene glycol linker. Such a linker may be referred to as a heterobifunctional PEG linker or a homobifunctional PEG linker.

In certain embodiments, a linker further comprises one or more spacers. Spacers are known in the art. Non-limiting examples of spacers include 2-aminoethoxy-2-ethoxy acetic acid (AEEA) linkers, AEEEA linkers, and AEA linkers. In a specific embodiment, a linker further comprises one or more 2-aminoethoxy-2-ethoxy acetic acid (AEEA) linkers.

Formulation

The agents and compositions described herein can be formulated in any conventional manner using one or more pharmaceutically acceptable carriers or excipients. Such formulations will contain a therapeutically effective amount of a biologically active agent described herein, which can be in purified form, together with a suitable amount of carrier to provide the form for proper administration to the subject.

“Formulation” refers to preparing a drug in a form suitable for administration to a subject, such as a human. Thus, a “formulation” can include pharmaceutically acceptable excipients, including diluents or carriers.

“Pharmaceutically acceptable” refers to substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components not described in the USP/NF may also be used.

The term “pharmaceutically acceptable excipient,” as used herein, can include all solvents, dispersion media, coatings, antibacterial and antifungal agents, and isotonic or absorption-delaying agents. The use of such media and agents for pharmaceutically active substances is well-known in the art. Except insofar as any conventional media or agent is incompatible with an active ingredient, its use in therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes, which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently, affect the occurrence of side effects. Controlled-release preparations may initially release an amount of an agent(s) that produces the desired therapeutic effect and gradually and continually release other amounts of the agent to maintain the therapeutic effect over an extended period. To maintain a near-constant level of an agent in the body, the agent can be released from the dosage form at a rate that will replace the amount of the agent being metabolized or excreted from the body. Various inducers may stimulate the controlled release of an agent, e.g., a change in pH, temperature, enzymes, water, or other physiological conditions or molecules.

Agents or compositions described herein can also be used with other therapeutic modalities, as described further below. Thus, in addition to the therapies described herein, one may also provide to the subject other therapies known to be efficacious for treating the disease, disorder, or condition.

Therapeutic Methods

Also provided is a process of treating, preventing, or reversing a pANXA2-mediated disorder in a subject in need of administration of a therapeutically effective amount of an LS301 agent to treat a pANXA2-mediated disorder, prevent progression of a pANXA2-mediated disorder, induce regression of a pANXA2-mediated disorder. pANXA2-mediated disorders include, but are not limited to, a joint disease, diabetes, inflammation, cardiovascular disease, and cancer.

The present disclosure provides a method of detecting a joint disease, the method comprising administering a composition comprising an agent chosen from LS301, LS838, or a derivative thereof to a subject in need thereof and detecting a signal intensity emitted from the agent in at least one joint of the subject, wherein detection of signal intensity above a baseline value indicates the subject has the joint disease.

The present disclosure provides a method of binding phosphorylated annexin A2 (pANXA2) protein in a biological sample comprising contacting the biological sample with a pharmaceutical composition disclosed herein. In certain embodiments, the binding is selective over annexin A1 (ANXA1), non-activated ANXA2, and annexin A3 (ANXA3).

Annexin A2, also known as annexin II, is a protein that, in humans, is encoded by the ANXA2 gene. Annexin A2 is involved in diverse cellular processes such as cell motility (especially that of the epithelial cells), linkage of membrane-associated protein complexes to the actin cytoskeleton, endocytosis, fibrinolysis, ion channel formation, and cell-matrix interactions. It is a calcium-dependent phospholipid-binding protein whose function is to help organize the exocytosis of intracellular proteins to the extracellular domain. Annexin A2 is a pleiotropic protein, meaning its function depends on place and time in the body. Annexin A2 has been shown to interact with Prohibitin, CEACAM1, S100A10, PCNA, complement Factor H, and several viral factors, including the HPV16 minor capsid protein L2. Exosome-associated Annexin A2 supports angiogenesis and breast cancer metastasis.

Annexin A2 contributes to lung injury and fibrotic disease by mediating the fibrogenic actions of FXa. Annexin A2 and HB-EGF are overexpressed and secreted into serum in Her-2 negative breast cancer patients. In inflammatory dendritic cells, ANXA2 preserves late endosomal/lysosomal membrane integrity, thus modulating inflammation in arthritis. Under homeostatic conditions, ANXA2 is anti-inflammatory in response to injury or infection. In the immediate response to injury, ANXA2 maintains vascular integrity, thereby preventing edema and extravasation of blood cells. Annexin A2 binds to endosomes and negatively regulates TLR4-triggered inflammatory responses via the TRAM-TRIF pathway.

In certain embodiments, the disease is chosen from stroke, myocardial infarction, deep vein thrombosis, and pulmonary embolism. In certain embodiments, the disease is kidney disease, optionally kidney disease resulting from acute tubular necrosis.

In certain embodiments, the disease is von Willebrand disease (VWD), a common hereditary blood-clotting disorder in humans arising from deficient von Willebrand factor. The three hereditary VWDs are VWD type 1, VWD type 2, VWD type 3, and various subtypes.

Also provided is a process of treating, preventing, or reversing a joint disease in a subject in need of administration of a therapeutically effective amount of an LS301 agent to treat a joint disease, prevent progression of a joint disease, induce regression of a joint disease, or reduce swelling, erythema, or inflammation in joints. “Joint disease” or “joint disorder” refers any disease, disorder, infection, or injury affecting or causing inflammation at a joint. Examples of joint diseases include, but are not limited to, arthritis, lupus, gout, bursitis, tendonitis, scoliosis, Sjögren's syndrome, sprain, strain, dislocated joint, or bone fracture.

“Arthritis” refers to inflammation of the joint. Examples of arthritis include, but are not limited to, osteoarthritis, hip arthritis, knee arthritis, rheumatoid arthritis, spondylarthritis (also known as spondylitis), and juvenile idiopathic arthritis. In certain embodiments, the disease is inflammatory arthritis, such as ankylosing spondylitis, gout, pseudogout, Lyme disease, lupus, psoriatic arthritis, and rheumatoid arthritis. In certain embodiments, the arthritis is osteoarthritis. In certain embodiments, the arthritis is rheumatoid arthritis.

In certain embodiments, the method reduces a symptom of a joint disease, for example, joint stiffness, decreased range of motion, decreased joint function, bumps on small finger joints, swelling of wrists and hands, large knuckles, persistent morning joint stiffness, fatigue, whole-body symptoms, bone pain at rest, joint pain, gradual loss of height or stooped posture, unexplained back pain, a shoulder or hip that's higher than the other, and leg-length discrepancies.

Methods described herein are generally performed on a subject in need thereof. A subject in need of the therapeutic methods described herein can be a subject having, diagnosed with, suspected of having, or at risk for developing a joint disease. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be human.

Generally, a safe and effective amount of an agent is, for example, an amount that would cause the desired therapeutic effect in a subject while minimizing undesired side effects. In various embodiments, an effective amount of an agent described herein can substantially inhibit swelling, erythema, or inflammation of joints, slow the progress of a joint disease, or limit developing a joint disease.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

In certain embodiments, the administration is intravenous. In certain embodiments, the detecting step is 12 to 48 hours after the administering step, for example about 12, 16, 20, 24, 28, 32, 36, 40, 44, or 48 hours after the administering step. In other embodiments, the detecting step occurs soon after the administering step, such as between about 1 and 3 hours.

In certain embodiments, the detecting step is performed by a mobile boom based imaging system that moves along the subject's body with a focus on the site of suspected disease. For example for a joint disease, the detecting step focuses on the subject's joints. Suitable systems include the Xiralite® Fluorescence Imaging System, which allows scanning of parts of the body in a box. This approach is especially adapted for the hands, feet, and possibly the elbows and knees. The disclosed methods can be used with any commercially available fluorescence imaging systems. One of skill in the can select other imaging systems. Likewise, other imaging modes can be adapted for other body parts.

In certain embodiments, the detecting step is performed via a handheld imaging system.

In certain embodiments, the method further comprise generating a three-dimensional image.

In certain embodiments, the detecting step occurs from an excitation in the near-infrared or short wavelength infrared. In certain embodiments, the emitted signal is a fluorescence signal.

When used in the treatments described herein, a therapeutically effective amount of an agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a joint disease, prevent the progression of a joint disease, induce regression of a joint disease, reduce swelling, erythema, or inflammation in joints.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not constitute a therapeutically effective amount, as the necessary therapeutically effective amount could be reached by administration of several individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀ (the dose therapeutically effective in 50% of the population). The dose ratio between toxic and therapeutic effects is the therapeutic index expressed as the ratio LD₅₀/ED₅₀, where larger therapeutic indices are generally understood in the art to be optimal.

The specific therapeutically effective dose level for any particular subject will depend upon a variety of factors, including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts. For example, it is well within the skill of the art to start doses of the composition at levels lower than those required to achieve the desired therapeutic effect and to gradually increase the dosage until the desired effect is achieved. If desired, the effective daily dose may be divided into multiple doses for administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions described herein and others can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the disease development or at least one clinical or subclinical symptom. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of an agent can occur as a single event or over a time course of treatment. For example, an agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

“Treatment” described herein can be performed before or before, concurrent with, or after conventional treatment modalities for a joint disease.

An agent can be administered simultaneously or sequentially with another agent, such as an antibiotic, an anti-inflammatory, or another agent. For example, an agent can be administered simultaneously with another agent, such as an antibiotic or an anti-inflammatory. Simultaneous administration can occur through the administration of separate compositions, each containing one or more of an LS301 agent, an antibiotic, an anti-inflammatory, or another agent. Simultaneous administration can occur by administering one composition containing two or more of an LS301 agent, an antibiotic, an anti-inflammatory, or another agent. An agent can be administered sequentially with an antibiotic, an anti-inflammatory, or another agent. For example, an agent can be administered before or after the administration of an antibiotic, an anti-inflammatory, or another agent.

Active compounds are administered at a therapeutically effective dosage sufficient to treat a condition for a condition in a patient. For example, the efficacy of a compound can be evaluated in an animal model system that may predict efficacy in treating the disease in a human or another animal, such as the model systems shown in the examples and drawings.

An effective dose range of a therapeutic can be extrapolated from effective doses determined in animal studies for various animals. In general, a human equivalent dose (HED) in mg/kg can be calculated per the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the practitioner's judgment and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, the severity of the condition, the type of disease being treated, previous or concurrent therapeutic interventions, idiopathy of the subject and on the route of administration. A skilled artisan may determine these factors. The practitioner responsible for administration will typically determine the concentration of active ingredient(s) in a composition and the appropriate dose(s) for the individual subject. The individual physician may adjust the dosage in the event of any complication.

In some embodiments, the agent may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, an agent such as a compound described herein may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may range from 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Administration

Agents and compositions described herein can be administered according to methods described herein in various means known to the art. The agents and composition can be used therapeutically as exogenous or endogenous materials. Exogenous agents are those produced or manufactured outside the body and administered to the body. Endogenous agents are those produced or manufactured inside the body by some device (biological or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in various methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition like that used for delivering insulin or chemotherapy to specific organs or tumors. Typically, using such a system, an agent or composition can be administered in combination with a biodegradable, biocompatible polymeric implant that releases the agent over a controlled time at a selected site. Polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes. Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve the taste of the product; or improve the shelf life of the product.

Kits

Also provided are kits. Such kits can include an agent or composition described herein and, in certain embodiments, instructions for administration. Such kits can aid the performance of the methods described herein. When supplied as a kit, the different components of the composition can be packaged in separate containers and admixed immediately before use. Components include, but are not limited to, an LS301 agent. If desired, such packaging of the components can be presented in a pack or dispenser device, which may contain one or more unit dosage forms containing the composition. The pack may, for example, comprise metal or plastic foil, such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and sterile saline in a separate ampule, sterile water, each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other suitable containers include bottles fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that a hypodermic injection needle can pierce. Other containers may have two compartments separated by a readily removable membrane that, upon removal, permits the components to mix. Removable membranes may be glass, plastic, rubber, and the like.

In certain embodiments, kits can be supplied with instructional materials. For example, instructions may be printed on paper or another substrate and/or supplied as an electronic-readable medium or video. In addition, detailed instructions for the kit may not be physical; instead, a user may be directed to an Internet website specified by the manufacturer or distributor of the kit.

A control sample or a reference sample, as described herein, can be a sample from a healthy subject or sample, a wild-type subject or sample, or from populations thereof. A reference value can be used in place of a control or reference sample previously obtained from a healthy subject, a group of healthy subjects, or a wild-type subject or sample. A control or reference sample can also be a sample with a known amount of a detectable compound or a spiked sample.

Compositions and methods described herein using molecular biology protocols can be according to various standard techniques known to the art.

Definitions and methods described herein are provided to better define the present disclosure and guide those of ordinary skill in the art practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” indicates that a value includes the standard deviation of the mean for the device or method employed to determine the value. In some embodiments, the numerical parameters set forth in the written description and attached claims are approximations that can vary depending on the desired properties obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors resulting from the standard deviation found in their respective testing measurements. The recitation of ranges of values herein is merely intended to serve as a shorthand method of referring individually to each separate value falling within the range. Unless otherwise indicated herein, each value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and can also cover other unlisted steps. Similarly, any composition or device that “comprises,” “has,” or “includes” one or more features is not limited to possessing only those one or more features and can cover other unlisted features.

All methods described herein can be performed in any suitable order unless otherwise indicated herein or otherwise clearly contradicted by context. The use of all examples or exemplary language (e.g., “such as”) provided concerning certain embodiments herein is intended merely to better illuminate the present disclosure and does not limit the scope of the present disclosure otherwise claimed. No language in the specification should be construed as indicating any non-claimed element essential to the practice of the present disclosure.

Groupings of alternative elements or embodiments of the present disclosure are not construed as limitations. Each group member can be referred to and claimed individually or combined with other group members or elements found herein. One or more group members can be included or deleted from a group for convenience or patentability reasons. When any such inclusion or deletion occurs, the specification is herein deemed to contain the group as modified, thus fulfilling the written description of all Markush groups used in the appended claims.

All publications, patents, patent applications, and other references cited in this application are incorporated herein by reference in their entirety for all purposes to the same extent as if each publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can constitute examples of modes for its practice. However, those of skill in the art should, in light of the present disclosure, appreciate that many changes can be made in the specific embodiments disclosed and still obtain a like or similar result without departing from the spirit and scope of the present disclosure.

### Example 1: Noninvasive Monitoring of Arthritis Treatment Response Via Targeting of Tyrosine-Phosphorylated Annexin A2 in Chondrocytes

In mouse models of spontaneous and serum transfer-induced inflammatory arthritis, intravenously administered LS301 showed selective accumulation in regions of joint pathology, including paws, ankles, and knees, with a positive correlation between fluorescent signal and disease severity by clinical scoring. Whole-body near-infrared imaging with LS301 allowed tracking of spontaneous disease remission and the therapeutic response after dexamethasone treatment. Histological analysis showed preferential accumulation of LS301 within the chondrocytes and articular cartilage in arthritic mice. Colocalization was observed between LS301 and pANXA2 in the joint tissue.

All the fluorenylmethyloxycarbonyl (Fmoc) amino acids and Fmoc-Lys (Boc)-Wang Resin were purchased from AAPPTec (Louisville, Ky., USA). Dichloromethane (DCM), acetic acid, acetic anhydride, thioanisole, phenol, hydroxybenzotriazole (HOBt), N,N-diisopropylethylamine (DIEA), N-trityl-1,2-ethanediamine, phenol, thioanisole, dimethylformamide (DMF), N,N′-diisopropylcarbodiimide (DIC), trifluoroacetic acid (TFA), iodine, methyl tert-butyl ether (MTBE), O-(7-azabenzotriazol-1-yl)-N,N,N′,N′-tetramethyluronium hexafluorophosphate (HATU), and dexamethasone (DEX) were purchased from Sigma-Aldrich (St Louis, Mo.). Hematoxylin and eosin (H&E) stains were purchased from MilliporeSigma (St Louis, Mo.). Rabbit antipANXA2 (phospho-Tyr24) antibody was purchased from Signalway Antibody (College Park, Md.). AlexaFluor 594-conjugated donkey antirabbit antibody was purchased from Thermo Fisher Scientific (Waltham, Mass.).

LS301 (cypate-cyclic (DCys-Gly-Arg-Asp-Ser-Pro-Cys)-Lys-OH) was synthesized as previously reported. Briefly, the linear GRD peptide, H-DCys (Acm)-Gly-Arg (Pbf)-Asp (tBu)-Ser (tBu)-Pro-Cys (Acm)-Lys (Boc)-OH, was prepared via a CEM Liberty Blue microwave peptide synthesizer (Matthews, N.C., USA) on the Fmoc-Lys (Boc)-wang resin. The resin (0.1 mmol) was swelled in DCM for 1 h before use. Fmoc-amino acids (0.5 mmol, 5 eq), coupling reagent (HBTU, 0.5 mmol, 5 eq), and DIEA (1 mmol, 10 eq) were added to the resin. The mixture was reacted for 15 min under microwave irradiation (100 W, 90° C.). The resin was washed three times with DMF.

Deprotection of Fmoc group was performed by treatment of 20% piperidine/DMF for 5 min under microwave irradiation (100 W, 90° C.). The peptidyl resin was washed. The peptide was cyclized through the disulfide bridge with iodine (1.2 eq) in DMF for 90 min. Subsequently, cypate (3 eq) was conjugated to the cyclic peptide on a solid support in the presence of DIC (5 eq) in DMF to afford the LS301 peptidyl resin. The resin was then treated with a cleavage cocktail of TFA:thioanisole:phenol:water (85:5:5:5, v/v/v/v) for 90 min at room temperature. The cleaved peptide product was concentrated in vacuo before purification by reverse-phase HPLC (Gilson, Middleton, Wis., USA). Analytical HPLC determined product purity (>95%). The compound identity was confirmed by electrospray ionization mass spectrometry on a Shimadzu LCMS-2020 Mass Spectrometer (Columbia, Md.) with peaks observed at 1470 (M+1) and 735 (M+2/2).

Male 5-7-week-old C57BL/6J mice were purchased from The Jackson Laboratory (Bar Harbor, Me.) and housed in designated animal facilities. Mice were fed ad libitum and inspected regularly.

The K/BxN mice with spontaneous arthritis (F1) were maintained in the laboratory. To establish serum transfer arthritis (STA), male 6-8 weeks old C57BL/6J mice were injected intraperitoneally with 150-175 μL of serum derived from F1 mice (8-9 weeks old). Day 0 denoted induction of serum transfer/disease. Clinical manifestation of arthritis in each paw was assessed daily on a scale of 0-3 with 0=no swelling or erythema, 1=slight swelling or erythema, 2=moderate erythema and swelling in multiple digits or entire paw, and 3=pronounced erythema and swelling of an entire paw, with a maximum score of 12 per mouse. The ankle thickness of two hind paws was measured using calipers. Animals were monitored for distress during arthritis induction, including their ability to move around the cage and access food/water.

Animals were shaved. Excess hair was removed using commercially available hair removal cream. Mice were anesthetized with isoflurane for injection and imaging procedures. LS301 stock (or LS301-methotrexate or LS301-methylprednisolone derivative stock) in dimethyl sulfoxide solution was diluted in phosphate-buffered saline to a final concentration of 60 μM and injected via tail vein into mice. In-vivo near-infrared fluorescence using 785 nm excitation and 820 nm emission filters was assessed pre-injection, post-injection, and/or at indicated time points post-injection with a Pearl Small Animal Imaging System (LICOR Biotechnology, Lincoln, Nebr.). Regions of interest (ROIs) for fluorescence quantitation were drawn and analyzed using the Pearl Small Animal Imaging System software.

Experiments were blinded, where the technician responsible for clinical assessment of paw score and ankle measurements was blinded to treatment groups. For studies on disease remission, mice with STA were injected with 6 nmol of intravenous LS301 on day 4 post-disease induction and imaged at 18 h post LS301 injection using the Pearl Small Animal Imaging System. Clinical paw scores and ankle measurements were obtained daily.

On day 23, post-disease induction, when the clinical paw scores of mice were near the baseline, mice were imaged again with 6 nmol intravenous LS301. Regions of interest (ROI) were quantitated, encompassing mouse upper extremities (all structures including and distal to the wrist) and lower extremities (all structures including and distal to the ankle), applied universally to all images using the Pearl software. Total extremity fluorescence (quantitated from ROIs) per mouse, averaged among n=3 mice, was compared between groups. For studies on response to DEX treatment, mice with STA were injected with 6 nmol of intravenous LS301 on day 3 post-disease induction and imaged at 18 h post LS301 injection using the Pearl Small Animal Imaging System with λ=785 nm (excitation)/820 nm (emission). Mice received intraperitoneal DEX (10 mg/kg/dose) daily over 6 days. Clinical paw scores and ankle measurements were obtained daily. On day 9 (the day of the final DEX treatment), mice were imaged again with 6 nmol intravenous LS301.

H&E staining, immunohistochemical staining for pANXA2, and microscopic analysis were performed. Tissues of interest were harvested and frozen at −80° C. in Optimal Cutting Temperature (OCT) media. Frozen sections were cut at 10 μm thickness, and slides were stored at −40° C. Consecutive sections were subjected to H&E and immunohistochemical analysis as follows. Frozen sections were fixed for 10 min in 4% paraformaldehyde solution (Sigma, St. Louis, Mo., USA) and stained with Harris hematoxylin for 90 s and with eosin (Sigma, St. Louis, Mo.) for 15 s, and then washed with tap water for 5 min. Some sections were stained with Safranin 0 and Fast Green counterstain (Musculoskeletal Histology and Morphometry Core, Washington University School of Medicine, St. Louis, Mo. For immunohistochemistry, slides were blocked with appropriate serum for 35 min or with 5% nonfat milk PBS (pH 7.4) overnight at 4° C. and incubated with primary antibody overnight at 4° C. or 1 h at 37° C. For pANXA2 studies, tissue sections were incubated with 1:250 rabbit anti-pANXA2 (phospho-Tyr24) antibody (Signalway Antibody, College Park, Md.). After washing twice with PBS, the tissue sections were incubated with 1:1000 AlexaFluor 594-conjugated donkey antirabbit antibody (Thermo Fisher Scientific, Waltham, Mass.) for 1 h at 25° C., respectively. Slides were rewashed and stained with DAPI nuclear stain for 5 minutes (Thermo Fisher Scientific, Waltham, Mass.) for 45 minutes at 37° C. After the final washes, a coverslip with aqueous fluorescence-saving mounting media was applied before imaging. Slides were viewed using an Olympus B61 epifluorescence microscope (Olympus Corp., Tokyo, Japan) with filters/channels as follows: DAPI (Ex/Em=330-385/420 nm), FITC (Ex/Em=460-500/510-560 nm), Texas Red (Ex/Em=542-582/604-644 nm), cypate (Ex/Em=750-800/818-873 nm), using exposure times 1 to 30 s and sensitivity settings ISO200-ISO1600, with the same parameters used for control and treatment groups. ImageJ software (National Institutes of Health, Bethesda, Md., USA) was used for image processing.

Tissues were homogenized using an ultrasonic processor in RIPA buffer (20 mM Tris-HCl, pH 7.5, 150 mM NaCl, 1 mM Na₂EDTA, 1 mM EGTA, 1% NP-40, 1% sodium deoxycholate, 2.5 mM sodium pyrophosphate, 1 mM b-glycerophosphate, 1 mM Na₃VO₄, 1 μg/ml leupeptin, 1 mM PMSF). The tissue lysates were clarified by centrifugation. The protein was denatured in SDS gel-loading buffer (100 mM Tris-HCl, 200 mM DTT, 4% SDS, 0.2% bromophenol blue, and 20% glycerol) at 95° C. for 10 min and then separated on 12% SDS-polyacrylamide gels (50 μg of the tissue protein per sample). After electrophoresis, proteins were transferred to PVDF membrane using an EC140 Mini Blot Module (Thermo EC, Holbrook, N.Y.) apparatus. The membrane was blocked for 1 h at room temperature in PBS containing 5% nonfat dry milk (w/v), 0.1% (v/v) Tween-20 (PBS-T), followed by incubation with Annexin A2 rabbit mAb (1:2000; Cat. 8235, Cell Signaling Tech.) or p-Annexin A2 mouse mAb (1:500; sc-135753, Lot #J2920; Santa Cruz) in PBS-T containing 3% nonfat dry milk (w/v) at 4° C. overnight. After washing three times for 10 min each in PBS-T, the membrane was incubated for 1 h with diluted polyclonal goat antirabbit IgG or polyclonal goat anti-mouse IgG conjugated to horseradish peroxidase in PBS-T containing 3% nonfat dry milk (w/v). The membrane was then washed three times for 10 min each in PBS-T and developed using the chemiluminescence ECL kit (Pierce) according to the manufacturer's instructions.

Differences between sample means were analyzed by a two-tailed unpaired t-test (Microsoft Excel) with p<0.05 as the threshold for statistical significance. Correlations between fluorescence measurements, clinical paw scores, and ankle thickness changes were analyzed using Pearson correlation (Microsoft Excel). For semiquantitative fluorescence analysis, 3 mice per group allow 80% power to detect an effect size of 1.67 by 2-sided 2-sample t-test at alpha=5%.

LS301 localizes to sites of joint inflammation in models of RA. In the K/BxN (F1) mice with spontaneous arthritis, inflammation occurs progressively in the paws, ankle, and knee joints leading to measurable local erythema and swelling. F1 mice with severe arthritis (9-10 weeks old) were injected intravenously with LS301 and imaged 18 h post-injection for near-infrared fluorescence using the Pearl Small Animal Imaging System.

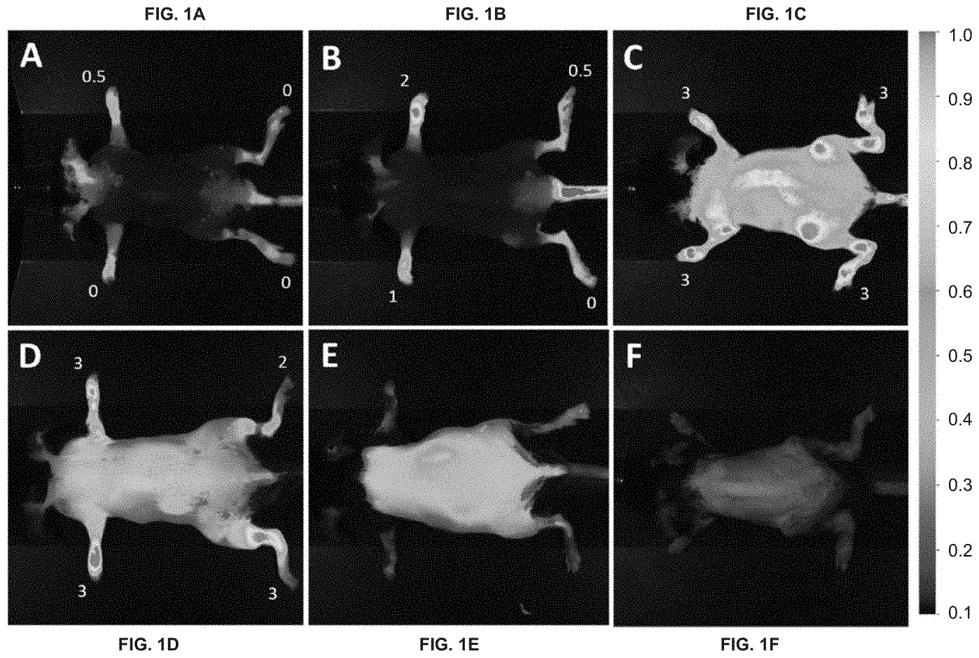

A time course assessment revealed the time point of contrast at ˜18 h post-injection when LS301 was seen to accumulate in regions of expected joint pathology in the extremities, including paws and ankles. Using this time point, fluorescence imaging using LS301 in F1 mice with early (3-4 weeks old), intermediate (5-7 weeks old), or late-stage arthritis (9-10 weeks old) showed a positive correlation between fluorescence signal and disease severity as assessed by clinical paw scoring (FIG. 1A-C). Similar results were obtained in mice with K/BxN serum-transfer arthritis (STA) (FIG. 1D).

FIG. 1(A-F) shows in-vivo imaging of K/BxN spontaneous and serum transfer arthritis using LS301. Arthritic or control (non-diseased) mice were injected intravenously with 6 nmol LS301. Whole-body near-infrared fluorescence images were taken on the Pearl animal imaging system with λ=785 nm excitation and λ=820 nm emission filters. Images shown represented at least two independent experiments and were taken at 18 h post-injection with mice in dorsal orientation. FIG. 1A shows early-stage spontaneous K/BxN arthritis (3-4 weeks old F1 mice). FIG. 1B shows intermediate-stage spontaneous K/BxN arthritis (5-7 weeks old F1 mice). FIG. 1C shows late-stage spontaneous K/BxN arthritis (9-10 weeks-old F1 mice). FIG. 1D shows C57BL/6J mice with serum transfer arthritis (day 4 post-disease induction). FIG. 1E shows normal (control) C57BL/6J mice injected with LS301. FIG. 1F shows intermediate-stage spontaneous K/BxN arthritis (5-7 weeks old) F1 mice injected with cypate dye (control) instead of LS301. Numbers denote individual clinical paw scores at the time of imaging.

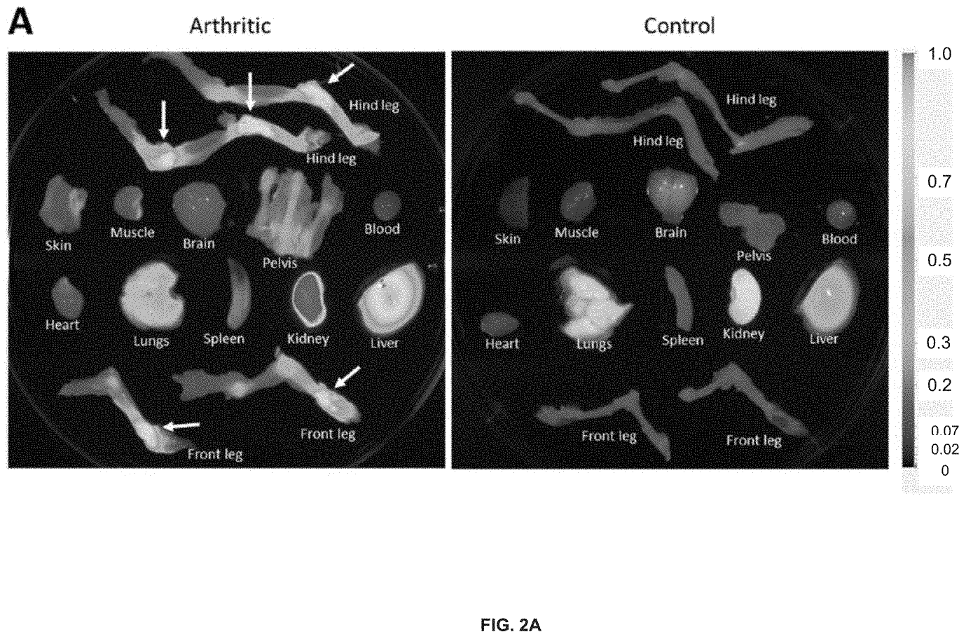

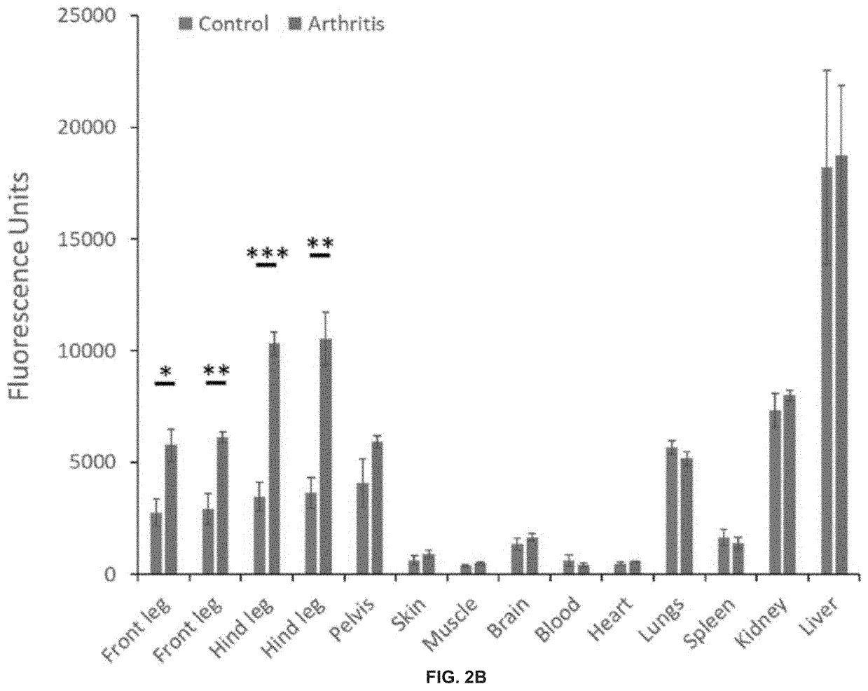

Ex-vivo biodistribution studies of LS301 in mice with induced arthritis confirmed selective accumulation of the agent in ankle and paw regions, with total fluorescence in individual limbs comparable to that of organs of excretion (liver, kidney) (FIG. 2). In contrast, healthy (non-diseased) control mice showed minimal LS301 accumulation in extremities (FIGS. 1E and 2). To exclude increased blood flow/impeded circulation as the primary factor leading to compound accumulation in the diseased sites, the cypate dye alone (the dye component of LS301) was administered to F1 arthritic mice (3-4 weeks old). No accumulation was found in extremities at 18 h (FIG. 1F). LS301 fluorescence correlates with arthritis disease severity in affected limbs.

FIG. 2 shows typical LS301 biodistribution in arthritic mice. FIG. 2A shows an example of ex-vivo organ biodistribution of LS301 in C57BL/6J mice with serum transfer arthritis (left) or in control C57BL/6J mice (right) 18 hours after intravenous injection, as assessed by near-infrared fluorescence imaging on the Pearl animal imaging system. C57BL/6J mice with serum arthritis (days 5-6 post serum transfer) (left) or control C57BL/6J mice (right) were injected intravenously with 6 nmol LS301. Organs were harvested at 18 hours post-injection. Near-infrared fluorescence images were taken on the Pearl animal imaging system with λ=820 nm. Arrows denote detected areas of joint inflammation. FIG. 2B quantifies fluorescence from individual organs from FIG. 2A (n=4 mice per group). ROIs were drawn around each organ/limb of interest and quantitated using the Pearl animal imaging system software.

The utility of LS301 as an imaging modality was assessed for monitoring disease activity. In a cohort of mice with STA, total fluorescence in each affected limb area encompassing the animal's upper extremities (all structures including and distal to the wrist) or lower extremities (all structures including and distal to the ankle), as determined using the Pearl software via ROI quantification, was plotted against clinical paw scores and ankle thickness measurements.

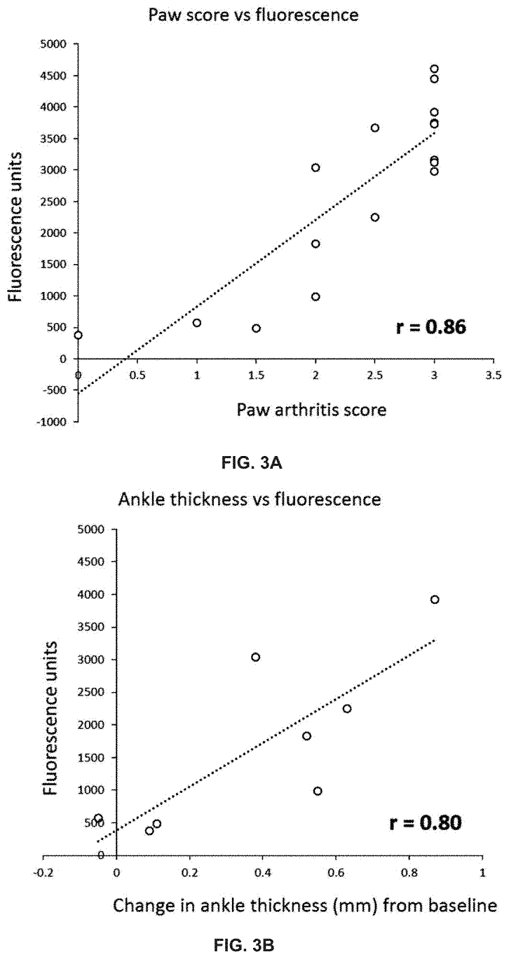

FIG. 3 correlates LS301 fluorescent signal with disease severity in individual limbs. C57BL/6 mice (n=4 per group) with serum transfer arthritis (day 4 post-disease induction) were injected intravenously with 6 nmol LS301. Whole-body near-infrared fluorescence images were taken on the Pearl animal imaging system with λ=820 nm. ROIs were drawn to quantitate LS301 near-infrared fluorescence in each limb (paws and ankles). Individual limbs of the mice were scored for paw edema and measured for ankle thickness by calipers. Fluorescence was plotted against clinical scores (FIG. 3A) and ankle thickness measurements (FIG. 3B). Pearson's correlation coefficient was calculated by the standard equation using Microsoft Excel software. LS301 fluorescence correlated positively with disease severity by both parameters (r=0.86 and r=0.80 respectively).

In addition, the LS301 fluorescence signal successfully discriminated between diseased and healthy (non-diseased) extremities using a threshold total clinical paw score of 1. These results demonstrate the potential of LS301 as a valuable tool for monitoring the severity of disease activity and progression.

Current paradigms in RA management generally require trial periods over months before a patient's therapeutic response to DMARDs can be determined via clinical scoring, imaging, and/or inflammatory markers. This delay leads to increased risks of disease progression, unnecessary drug toxicities, and financial expenses. Therefore, an imaging technique capable of reporting early response to drug treatment would have a significant translational impact.

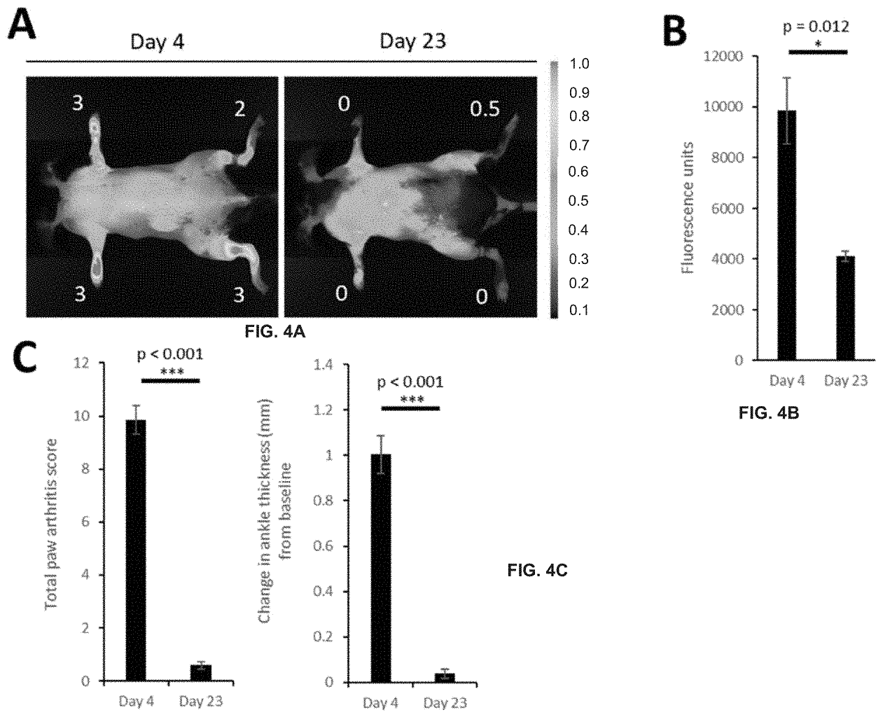

LS301 administration alone at the imaging dose (6 nmol) did not significantly affect disease progression. As shown in FIG. 4, LS301 fluorescence correlated with disease progression and regression in these mice, as orthogonally confirmed by clinical paw scoring. FIG. 4A shows the representative fluorescence images of arthritic mice imaged with LS301 on day 4 (left) and after disease remission (right). C57BL/6 mice (n=3 per group) with induced arthritis (STA) were imaged with 6 nmol intravenous LS301 at day 4 post-disease induction. At day 23 post-disease induction, when the clinical paw scores of mice were near the baseline, mice were imaged again with 6 nmol intravenous LS301. Whole-body near-infrared fluorescence images were taken on the Pearl animal imaging system with λ=820 nm. Images were taken 18 hours post-injection with mice in dorsal orientation and depicted representative independent replicates. Numbers denote individual clinical paw scores at the time of imaging. FIG. 4B shows the comparison of average total extremity fluorescence (quantitated from ROIs) per mouse described in FIG. 4A. FIG. 4C shows the total paw arthritis scores and change in ankle thickness from the baseline of mice described in FIG. 4A.

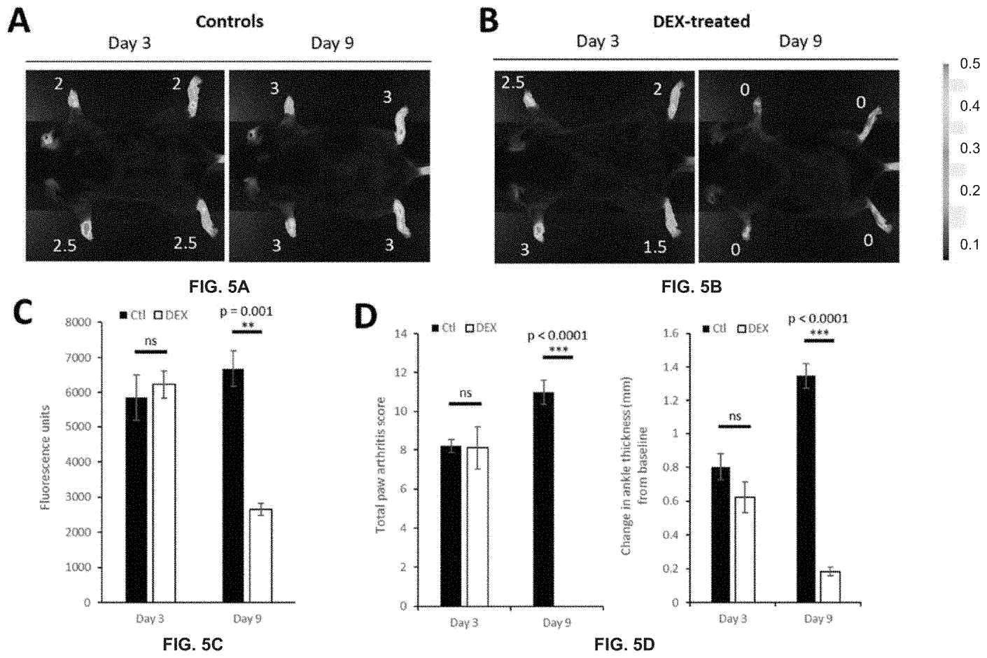

Next, to evaluate the use of LS301 in monitoring early treatment response, mice with STA (day 3 post-disease induction) were imaged using LS301. FIG. 5 monitors DEX-associated treatment response using LS301. Representative fluorescence images of arthritic mice imaged with LS301 before (left) and after (right) dexamethasone (DEX) treatment. C57BL/6 mice with induced arthritis (STA) (n=3 per group) were imaged with 6 nmol intravenous LS301 at day 3 post-disease induction. Mice in FIG. 5A remained untreated as controls or FIG. 5B were treated with daily doses of intraperitoneal dexamethasone (10 mg/kg/dose) over 6 days.

At day 9 post-disease induction, mice were imaged again with 6 nmol intravenous LS301. Whole-body near-infrared fluorescence images were taken on the Pearl animal imaging system with λ=820 nm. Images were taken at 18 hours post-injection with mice in dorsal orientation. Numbers denote individual clinical paw scores at the time of imaging. FIG. 5C compares the average total extremity fluorescence (quantitated from ROIs) per mouse described in FIGS. 5A and 5B. FIG. 5D shows the total paw arthritis scores and change in ankle thickness from the baseline of mice described in FIGS. 5A and 5B.

While control mice continued to show disease progression as assessed by a high level of LS301 fluorescence and clinical paw scores (FIG. 5A, C-D), mice treated with DEX showed reductions in disease severity that correlated closely with LS301 fluorescence (FIG. 5B, C-D). Taken together, these results demonstrate the utility of LS301 fluorescence for monitoring early treatment response for RA.

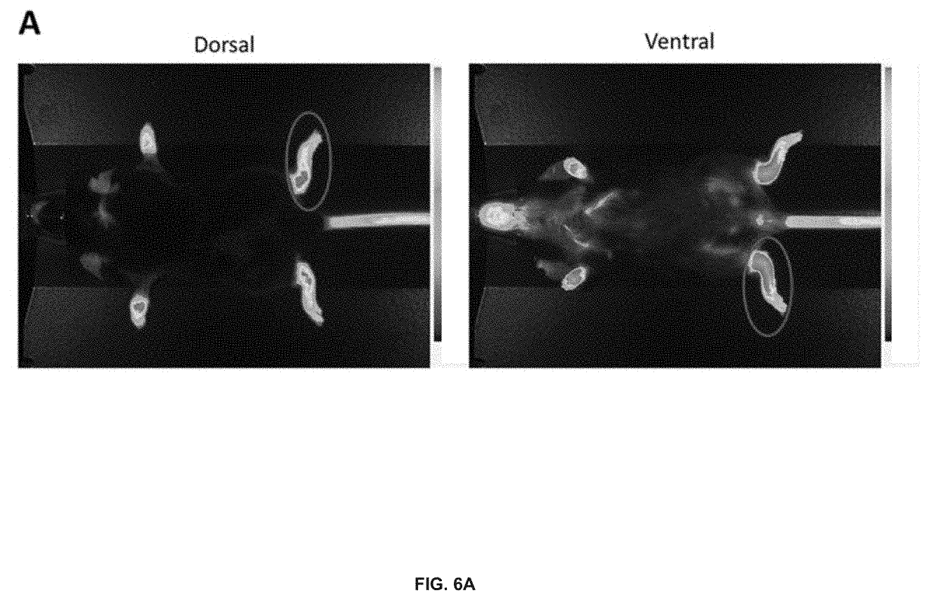

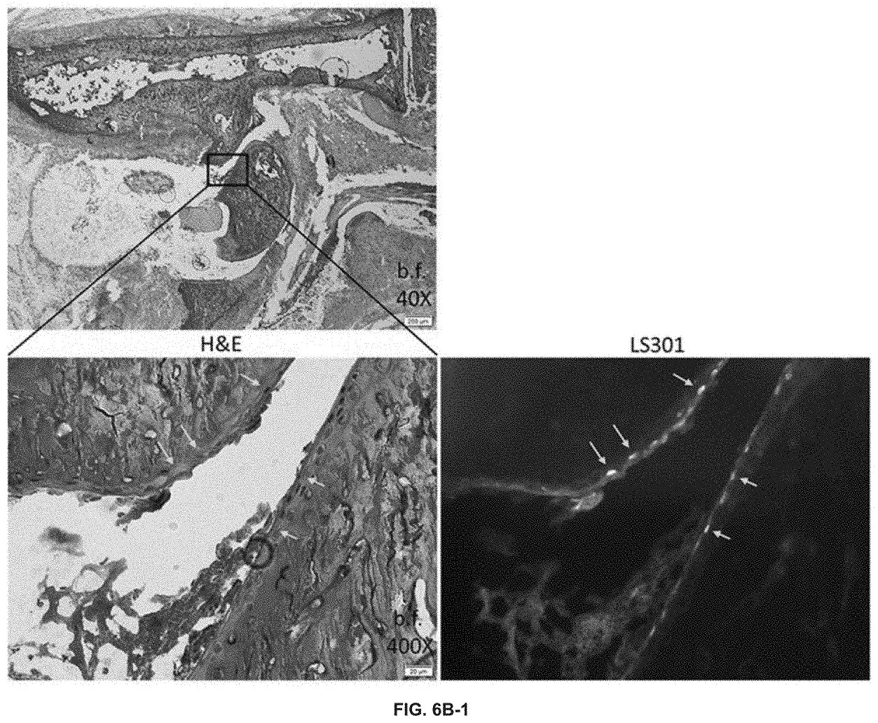

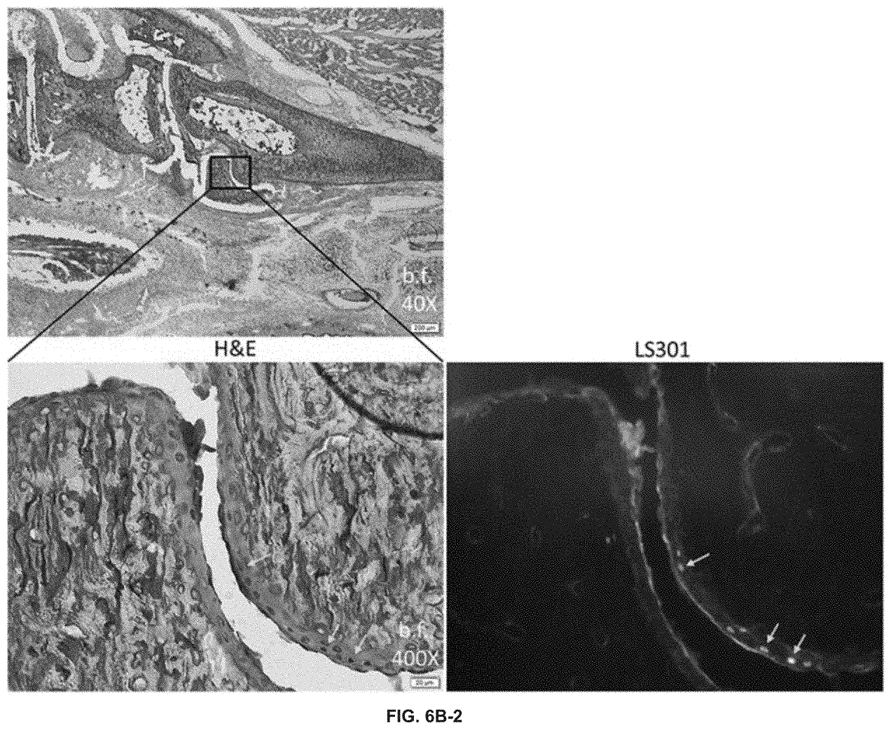

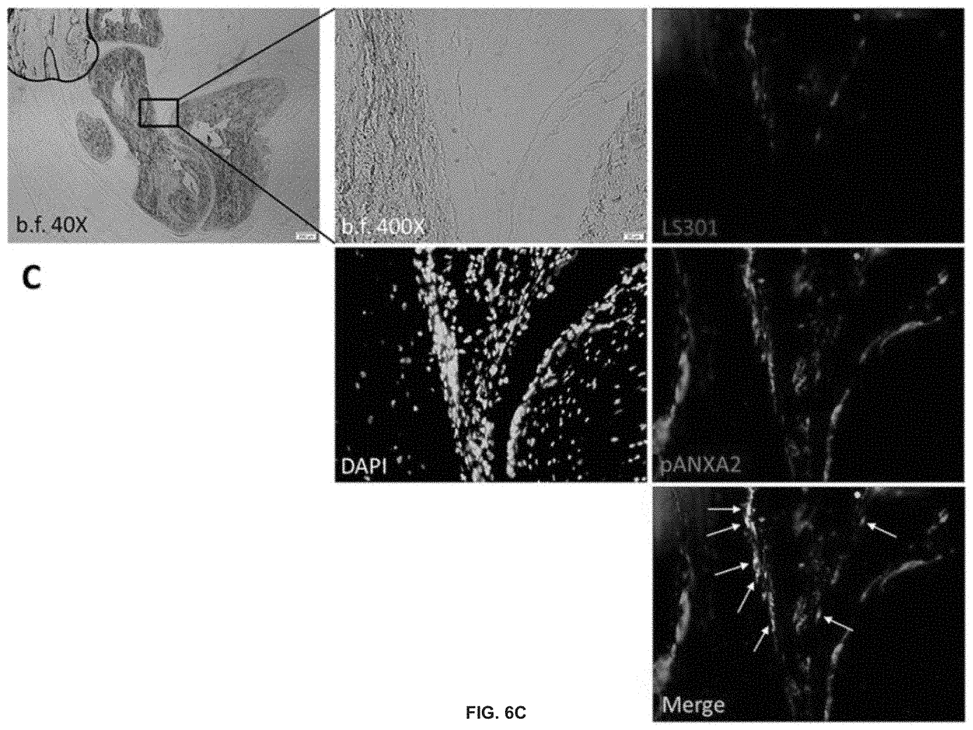

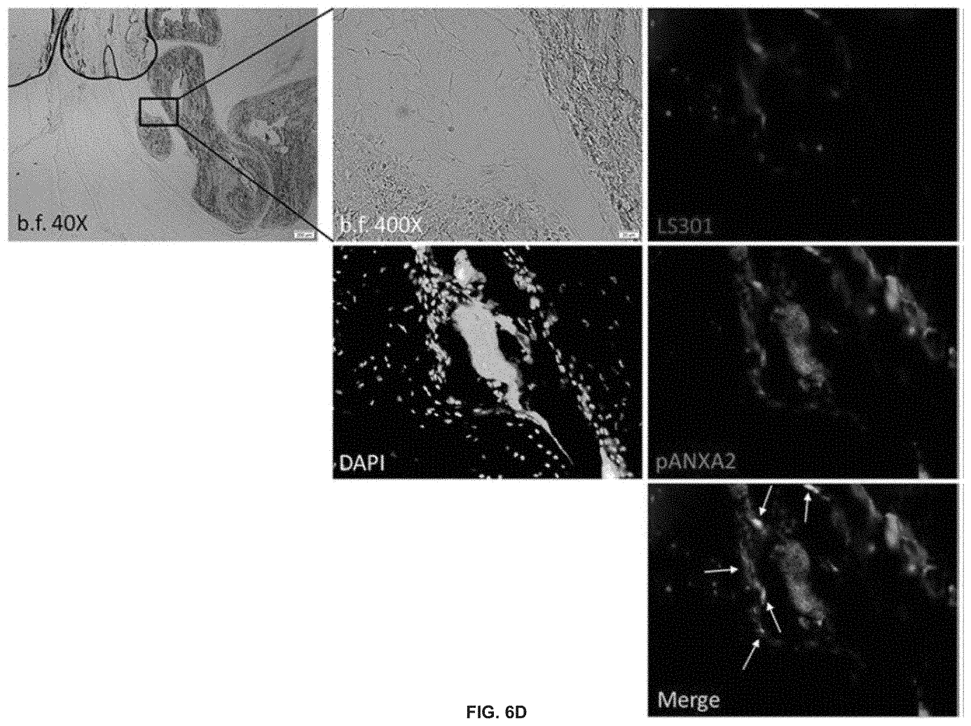

FIG. 6 shows the cellular localization of LS301 in the arthritic mouse paws. C57BL/6 mice with serum transfer arthritis were intravenously injected with 6 nmol LS301 at day 4 post-disease induction. 6 h after LS301 injection, whole-body near-infrared fluorescence images were taken on the Pearl animal imaging system with λ=820 nm. Subsequently, paws and ankles were harvested and frozen for sectioning. Sections were stained with H&E or left unstained and examined for LS301 fluorescence by microscopy under the cypate channel (Ex/Em 775±25 nm/845±28 nm) (red) and/or viewed for pANXA2 (AlexaFluor 594 fluorescence) (green) under the Texas Red channel (Ex/Em 562±20 nm/624±20 nm). Images represented results from at least two independent experiments. FIG. 6A shows an example of Pearl near-infrared fluorescence images of mice immediately before limb harvest. Red circles denote representative examples of the limb area harvested for sectioning. FIG. 6B(1-2) H&E and fluorescence images from corresponding regions of articular cartilage in mouse ankle. Arrows indicate example regions of LS301 accumulation. FIG. 6C shows fluorescence microscopy images from corresponding regions of articular cartilage in mouse ankles. Sections were stained with DAPI and anti-pANXA2 Ab/AlexaFluor 594-conjugated secondary Ab. Shown are DAPI (grayscale), LS301 fluorescence (red), and pANXA2 (green). Arrows denote LS301-pANXA2 colocalization.

LS301 accumulates within chondrocytes and articular cartilage in arthritic mice. To assess the tissue distribution of LS301, LS301 was administered to mice with STA and harvested mouse ankle tissues for fluorescent and immunohistochemical (IHC) analyses (FIG. 6A). H&E-stained sections of ankles showed precise localization of LS301 fluorescence within chondrocytes and articular cartilage by the superimposition of fluorescence images with H&E-stained images (FIG. 6B). In contrast, minimal or no LS301 signal above the background was detected in other tissue regions including skin, connective tissue, muscle, bone, or bone marrow. Previously, we found that LS301 binds with high affinity to pANXA2, which is known to be upregulated in arthritic cartilage. IHC staining of ankle sections for pANXA2 revealed significant colocalization of LS301 fluorescence with pANXA2 expression within articular cartilage (FIG. 6C).

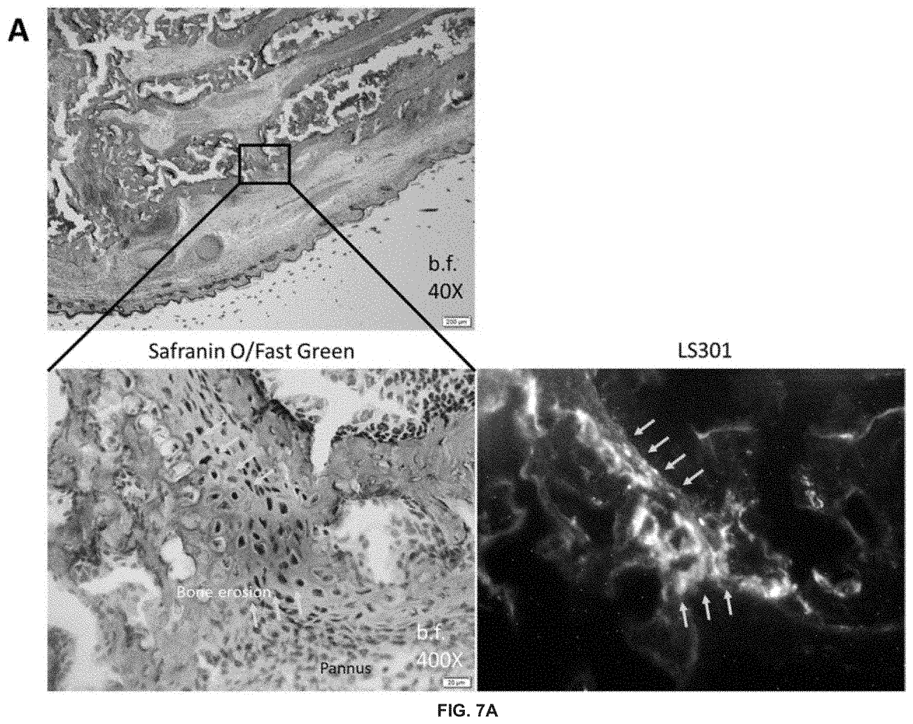

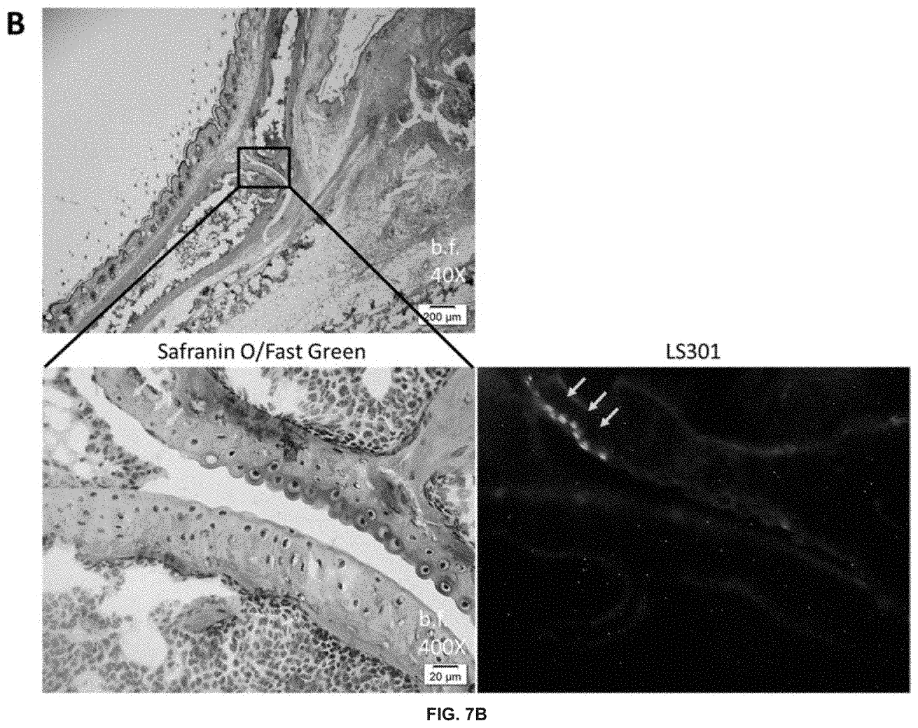

To further evaluate the correlation between cartilage damage and LS301 accumulation in the joint, we examined paw sections stained with Safranin 0. FIG. 7 shows LS301 association with cartilage damage. K/BxN F1 mice (6-7 weeks old) (n=2 mice per group) were injected intravenously with 6 nmol LS301. Six hours after LS301 injection, paws and ankles were harvested and frozen for sectioning. Sections were stained with Safranin 0/Fast Green and viewed for LS301 fluorescence by microscopy under the cypate channel (Ex/Em 775±25 nm/845±2 8 nm). FIG. 7A shows images from regions associated with pannus/bone erosion. Arrows indicate areas of LS301 accumulation. FIG. 7B shows images with a preferential association of LS301 at regions of cartilage damage (loss of Safranin 0 staining). Arrows indicate areas of LS301 accumulation. LS301 localizes to areas of pannus/bone erosion (FIG. 7A). Moreover, LS301 preferentially localizes to regions of damaged cartilage, as evidenced by loss of Safranin 0 staining (FIG. 7B). These results indicate that LS301 targets chondrocytes in areas of damaged cartilage.

LS301 localizes preferentially in chondrocytes within the arthritic articular cartilage (FIG. 6B). As the sole producers of cartilaginous matrix and mediators of inflammation, chondrocytes represent a future cellular target for therapy in arthritis, including RA and osteoarthritis. Chondrocytes have been a relatively overlooked target in fluorescence imaging approaches for RA, which typically focus on inflammatory cells such as macrophages. Thus far, efforts to deliver drugs to chondrocytes have had limited success due to the rapid clearance of molecules from the joint space following injections, the avascular nature of cartilage tissue and the location of chondrocytes in the relatively inaccessible middle and deep zones of cartilage. The observed penetration of LS301 into chondrocytes raises the possibility of specific drug delivery to these cells via conjugation with LS301. Further studies are warranted to elucidate the unique mechanism(s) enabling LS301 to traverse the biological barriers posed by the cartilaginous tissues.

In the biodistribution studies, some degree of natural inter-individual variability exists in organ LS301 uptake. For example, in a proportion of the mice, a possible nonsignificant trend toward increased kidney signal in arthritic mice when compared to controls was observed. G6PI-antibody immune complexes, which are a component in the pathogenesis of our serum transfer arthritis model, also localize to the kidney glomeruli. In some individuals, such a process could result in inflammation/elevated pANXA2 expression and LS301 accumulation in this region.

In line with a shift toward precision medicine, strategies for targeting drugs to sites of inflammation could enhance the potential of existing rheumatologic drugs by increasing local delivery and reducing off-target toxicity. Although intra-articular injection of therapeutics can achieve high local concentrations, this approach becomes impractical in cases involving multiple joints, such as in RA. LS301 accumulates in target inflammation areas following administration, suggesting its potential to circumvent these challenges. The chemical structure of LS301 enables it to readily serve as a covalent drug carrier via linkage with small molecule drugs or peptides.

Thus, FI using the pANXA2-targeting agent LS301 can monitor the progression, remission, and early response to drug treatment in mouse models of RA. The observed selectivity of LS301 for arthritic lesions and associating LS301 with chondrocytes in vivo provide a novel potential avenue for molecularly targeted imaging and drug evaluation.

### Example 2: Agent and System for Imaging and Therapy of Arthritis

This example describes targeted imaging and therapy of arthritis using LS301 and LS301 drug conjugates.

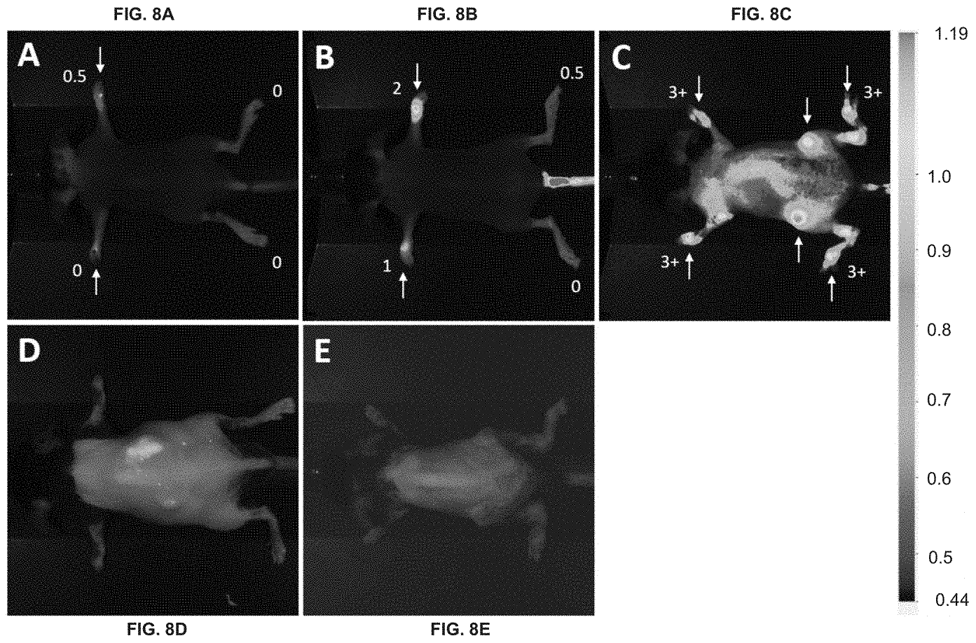

FIG. 8 shows imaging of rheumatoid arthritis (RA) using LS301. Mice were injected intravenously with 6 nmol LS301. Whole body near-infrared fluorescence images were taken on Pearl Imaging System with λ=800 nm. Images shown were taken at 18 h post-injection with mice in dorsal orientation. (A) Early stage RA (3-4 week old K/Bxn mice). (B) Intermediate stage RA (3-4 week old K/Bxn mice). (C) Late-stage RA (9-10 week old K/Bxn mice). (D) Normal (control) C57BL/6J mice. (E) K/Bxn mice (3-4 weeks old) were injected with cypate dye (control) instead of LS301. Arrows denote locations of detected joint pathology. Numbers denote individual clinical paw scores at the time of imaging.

LS301 selectively accumulated in regions of arthritic joint disease in the spontaneous K/Bxn model of RA, with fluorescence intensity correlating to disease severity. LS301 diagnostically identified animals with RA vs. healthy (non-diseased) animals.

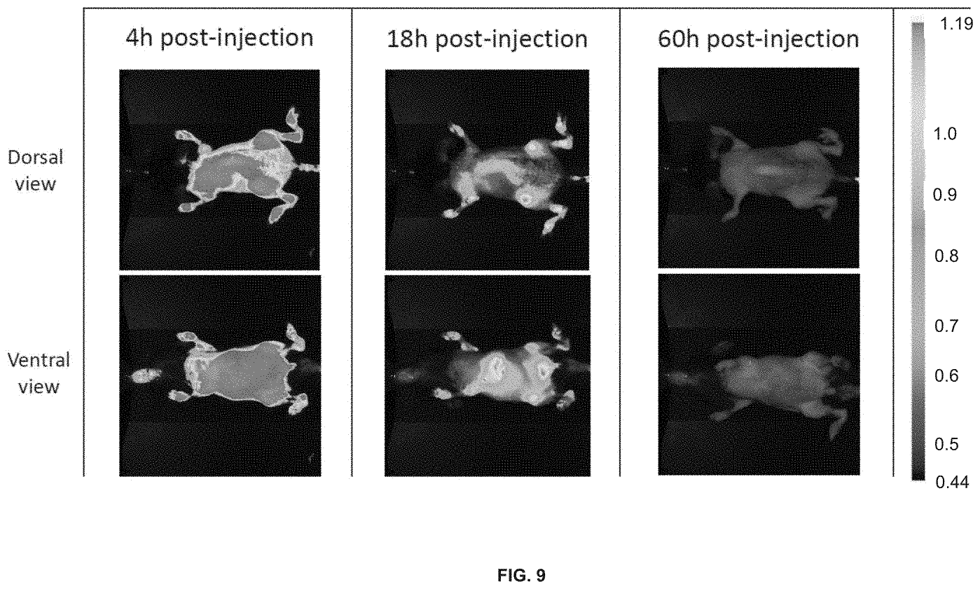

FIG. 9 shows the kinetics of in vivo LS301 clearance. K/BxN mice (9-10 wk. old) were injected intravenously with 6 nmol LS301. Whole body near-infrared fluorescence images were taken on Pearl Imaging System with λ=800 nm. LS301 accumulated in arthritic joints with maximum disease-specific contrast at 18 h, with clearance from the body after 2.5 days.

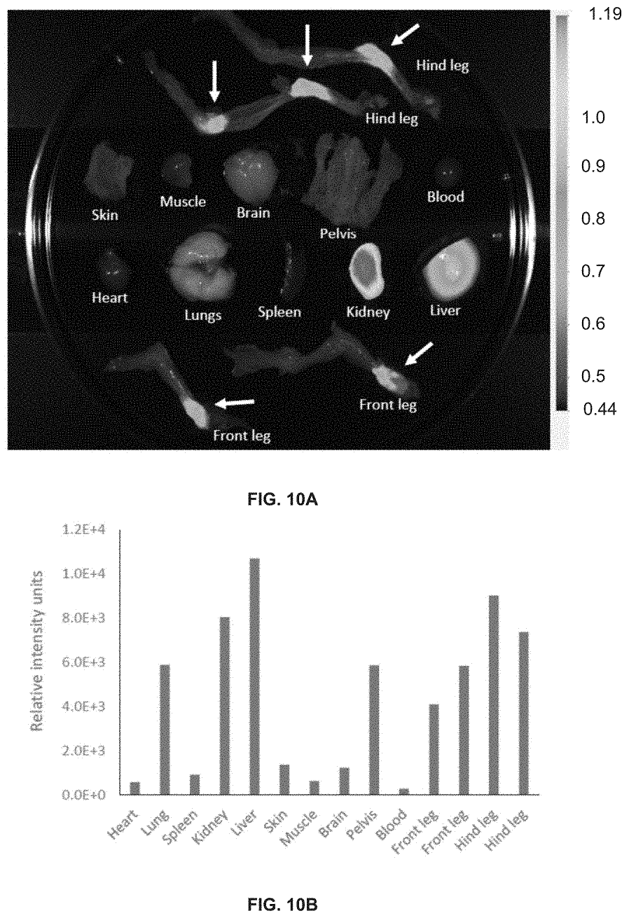

FIG. 10 shows typical LS301 biodistribution in arthritic mice. C57BL/6 mice with induced arthritis (K/Bxn serum transfer, days 5-6 post serum transfer) were injected subcutaneously (flank) with 6 nmol LS301. Organs were harvested at 18 h post-injection. Near-infrared fluorescence images were taken on Pearl Imaging System with λ=800 nm. LS301 showed substantial accumulation in arthritic joints at 18 h at a level comparable to organs of excretion (liver/kidney).

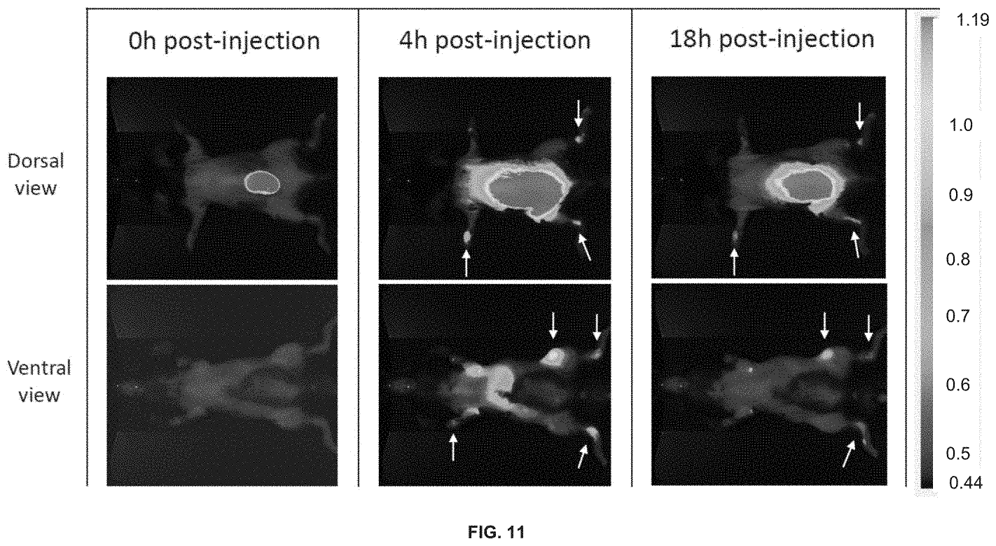

FIG. 11 shows imaging arthritis using subcutaneously administered LS301. C57BL/6 mice with induced arthritis (K/Bxn serum transfer, days 5-6 post serum transfer) were injected subcutaneously (flank) with 6 nmol LS301. Whole body near-infrared fluorescence images were taken on Pearl Imaging System with λ=800 nm. Arrows denote locations of detected joint pathology. LS301 can be administered subcutaneously, a translatable route of administration used in the outpatient clinical setting.

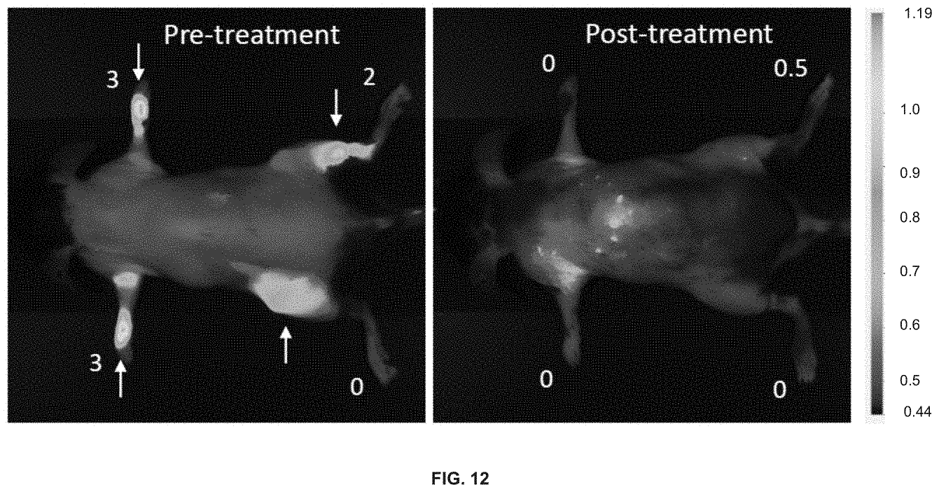

FIG. 12 shows the monitoring of arthritis remission using LS301. C57BL/6 mice with induced arthritis (K/Bxn serum transfer) were imaged with 6 nmol intravenous LS301 at day 4 post-disease induction. Mice were treated with three doses of intraperitoneal methotrexate (2 mg/kg/dose) over seven days. At day 23 post-treatment initiation (when clinical scores of mice had significantly decreased), mice were imaged again with 6 nmol intravenous LS301. Whole body near-infrared fluorescence images were taken on Pearl Imaging System with λ=800 nm. Images shown were taken at 18 h post-injection with mice in dorsal orientation. Arrows denote locations of detected joint pathology. Numbers denote individual clinical paw scores at the time of imaging. LS301 tracked arthritis disease remission after drug treatment.

Targeted therapy of arthritis using LS301 and LS301-drug conjugates:

(a) Concept #1: Delivery of an LS301-methotrexate conjugate with the release of drug b existing enzymes naturally present at disease sites
(b) Concept #2: LS301 combined with near-IR laser irradiation for therapeutic benefit via heat and reactive oxygen species generation

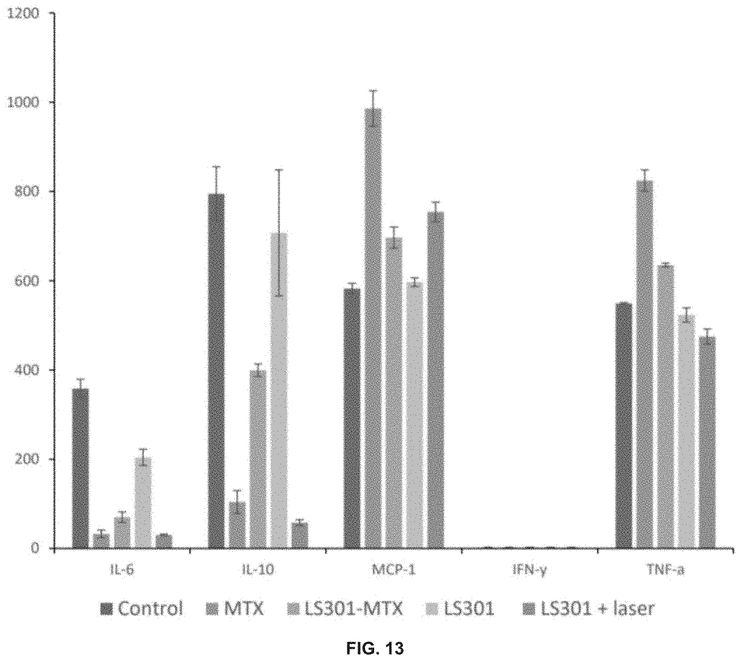

FIG. 13 shows the in-vitro anti-inflammatory effect of LS301-methotrexate (MTX) derivative, or LS301 combined with laser irradiation, vs. MTX alone and controls. Raw 264.7 macrophage cells were treated with methotrexate (MTX), LS301-MTX, LS301 alone, or LS301 combined with near-infrared (780 nm) laser irradiation. Levels of inflammation-associated (LPS-induced) cytokines, including IL-6 and IL-10, were measured by cytokine bead array. LS301-drug derivative and LS301 combined with laser irradiation suppress the inflammatory state of macrophages (a constituent and pathological cause of rheumatoid arthritis lesions).

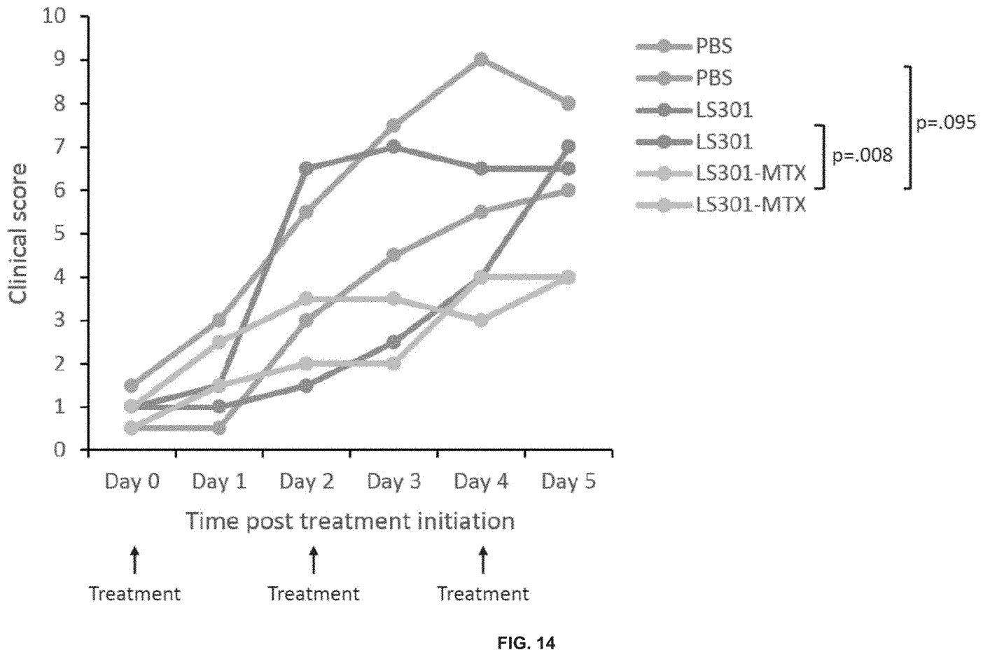

FIG. 14 shows the in-vivo therapeutic efficacy of LS301-MTX derivative vs. LS301 alone or control. C57BL/6J mice with induced arthritis (K/Bxn serum transfer, days 2-3 post-disease induction) were injected intraperitoneally with 88 nmol LS301-methotrexate (MTX) derivative, LS301 alone, or PBS (control) per treatment cycle. Individual limbs of the mice were scored for paw edema and measured for ankle thickness by calipers. The LS301-drug derivative showed a trend of therapeutic efficacy against rheumatoid arthritis in vivo.

What is claimed is:

1. A method of detecting a joint disease, the method comprising:
administering a composition comprising an agent chosen from LS301, LS838, or a derivative thereof to a subject in need thereof; and
detecting a signal intensity emitted from the agent in at least one joint of the subject, wherein detection of signal intensity above a baseline value indicates the subject has the joint disease.

2. The method of claim 1, wherein the administration is intravenous.

3. The method of claim 1, wherein the detecting step is 12 to 48 hours after the administering step.

4. The method of claim 1, wherein the detecting step is performed by a mobile boom based imaging system that moves along the subject's body with a focus on the joints.

5. The method of claim 1, wherein the detecting step is performed via a handheld imaging system.

6. The method of claim 1, further comprising generating a three-dimensional image.

7. The method of claim 1, wherein the detecting step occurs from an excitation in the near-infrared or short-wavelength infrared.

8. The method of claim 1, wherein the joint disease is arthritis, lupus, or multiple sclerosis.

9. The method of claim 8, wherein the joint disease is rheumatoid arthritis.

10. The method claim 1, wherein the emitted signal is a fluorescence signal.

11. The method of claim 10, wherein the emitted signal has a wavelength of about 820 nm.

12. A method for monitoring disease progression of a joint disease, the method comprising:
administering a composition comprising an agent chosen from LS301, LS838, or a derivative thereof to a subject in need thereof;
detecting a first signal intensity emitted from the agent in at least one joint of the subject at a first time point; and
detecting a second signal intensity emitted from the agent in at least one joint of the subject at a second time point, wherein
if the second signal intensity is less than the first signal intensity, the disease is determined to be regressing and if the second signal intensity is greater than the first signal intensity, the disease is determined to be progressing.

13. The method of claim 12, wherein the joint disease is arthritis, lupus, or multiple sclerosis.

14. A method of monitoring treatment response to a therapeutic agent for treating a joint disease in a subject in need thereof, the method comprising:
(i) administering a composition comprising an agent chosen from LS301, LS838, or a derivative thereof to the subject;
(ii) detecting a first signal intensity emitted from the agent in at least one joint of the subject at a first time point;
(iii) administering a therapeutic agent to the subject:
(iv) repeating step (i);
(v) detecting a second signal intensity emitted from the agent in at least one joint of the subject at a second time point; and
(vi) comparing the second signal intensity to the first signal intensity.

15. The method of claim 14, wherein the joint disease is arthritis, lupus, or multiple sclerosis.

16. The method of claim 14, wherein administering the therapeutic agent to the subject occurs less than about one week before detecting the second signal intensity emitted from the agent.

17. A method of treating a joint disease in a subject in need thereof, the method comprising; administering a composition comprising an agent chosen an LS301 derivative or an LS838 derivative comprising a therapeutic agent.

18. The method of claim 17, wherein the therapeutic agent is a disease-modifying antirheumatic (DMARD) or a steroid.

19. The method of claim 18, wherein the therapeutic agent is methotrexate, methylprednisolone, or dexamethasone.

20. The method claim 17, wherein the joint disease is arthritis, lupus, or multiple sclerosis.
